SEQUENCE LISTING (14 sequences)

SEQ ID NO: 1 - DNA, 22 bp, Artificial
  Primer
gtgggggtgactgagaagcagt

SEQ ID NO: 2 - DNA, 20 bp, Artificial
  Primer
aagctctgtcggctctggga

SEQ ID NO: 3 - DNA, 20 bp, Artificial
  Primer
gccctttttccgcttttgtg

SEQ ID NO: 4 - DNA, 19 bp, Artificial
  Primer
atcgccggtgcagtcatga

SEQ ID NO: 5 - DNA, 20 bp, Artificial
  Primer
tggaagcccgaggccttgtc

SEQ ID NO: 6 - DNA, 20 bp, Artificial
  Primer
agtggaagcccgaggccttg

SEQ ID NO: 7 - DNA, 2010 bp, Homo sapiens
caagaagtggcgaagttacctttgagggtatttgagtagcggcggtgtgtcaggggctaaagaggaggacgaagaaaagcagagcaagggaacccagggcaacaggagtagttcactccgcgagaggccgtccacgagacccccgcgcgcaggcatgagccccgccccccacgcatgagccccgccccccgctgttgcttggagaggggcgggacctggagagaggctgctccgtgaccccaccatgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctagggtggaaggggccctgggcgagcctgtagacctatcggctctcatcccttgggataagccccagtccaggtccaggaggctccctccctgcccagcgagtcttccagaaggggtgaaagggttgcaggtcccgaccactgacccttcccggctgccctccctccccagcttacacctcaagcccagcacgcagcgtaccttgaacagagggaggggaggacccatggctctccccccctagcccgggagaccaggggccttcctcttcctctgcttttatttaataaaaataaaaacagaaa

SEQ ID NO: 8 - DNA, 1528 bp, Homo sapiens
cagtgccttggtaatgaccagggctccagaaagagatgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatgactgttctgtccccatattttctgttttcttggccggatggctgaggcctggtggctgcctcctagaagtggaacagactgagaagggcaaacattcctgggagctgggcaaggagatcctcccgtggcattaaaagagagtcaaagggt

SEQ ID NO: 9 - DNA, 1964 bp, Mus musculus
ggcgaagttacttttgcttttcgctcagcaagcgctgttgcttcgagctactcccaggcttctgaagttacttccaaagtgctgtggaggcacaatcaccggtgcggacacagaggcaactctcgcctcccacggccgtttccagggcaacagagtcggagaccccctgcgacccccctcccgatcgccggtgcagtcatgagccccgcctccccctggtgcacggagaggggcggggcctggaacaagcaggctgcttcgtgacccactatgtcttcccccacaagttctctggacactcccgtgcctgggaatggttctcctcagcccagtacctccgccacgtcacccactattaaggaagaggggcaggagactgatcctcctccaggctctgaagggtccagctctgcctacatcgtggtcatcttagagccagaggatgagcctgagcgcaagcggaagaaggggccggccccgaagatgctgggccatgagctgtgccgcgtgtgcggagacaaggcctcgggcttccactacaacgtgctcagctgtgaaggctgcaaaggcttcttccggcgcagtgtggtccacggtggggccgggcgctatgcctgtcggggcagcggaacctgccagatggatgccttcatgcggcgcaagtgccagctctgccggctgcgcaagtgcaaggaggctggcatgcgggagcagtgcgtgctctctgaggagcagattcggaagaaaaggattcagaagcagcaacagcagcagccaccacccccatctgagccagcagccagcagctcaggccggccagcggcctcccctggcacttcggaagcaagcagccagggctccggggaaggagagggcatccagctgaccgcggctcaggagctgatgatccagcagttagttgccgcgcagctgcagtgcaacaaacgatctttctccgaccagcccaaagtcacgccctggcccctgggtgcagaccctcagtcccgagatgcccgtcagcaacgctttgcccacttcaccgagctagccatcatctcggtccaggagattgtggactttgccaagcaggtgccagggttcttgcagttgggccgggaggaccagatcgccctcctgaaggcgtccaccattgagatcatgttgctagaaacagccagacgctacaaccacgagacagaatgcatcacgttcctgaaggacttcacctacagcaaggacgacttccaccgtgcaggcttgcaggtggaattcatcaatcccatcttcgagttctcgcgggccatgcggcggctgggcctggacgatgcagagtatgccttgcttatcgccatcaacatcttctcagccgatcggcctaatgtgcaggagcccagccgtgtggaggccctgcagcagccatacgtggaggcgctcctctcctacacgaggatcaagcgcccacaggaccagctccgcttcccacgcatgctcatgaagctggtgagcctgcgcaccctcagctccgtgcactcggagcaggtctttgcattgcgactccaggacaagaagctgccgcccttgctgtccgagatctgggatgtgcacgagtaggggcagccacaagtgccccagccttggtggtgtcttcttgaagatggactcttcacctctcctcctggggtgggaggacattgtcacggcccagtccctcgggctcagcctcaaactcagcggcagttggcactagaaggccccaccccacccattgagtcttccaagagtggtgagggtcacaggtcctagcctctgaccgttcccagctgccctcccacccacgcttacacctcagcctaccacaccatgcaccttgagtggagagaggttagggcaggtggccccccacagttgggagaccacaggccctctcttctgccccttttatttaataaaaaaacaaaaataaa

SEQ ID NO: 10 - DNA, 1753 bp, Mus musculus
gggaacgctgactctggaggctgctgggattagggtgggggtgactgagaagcagtccttctgtcagagcaaagagcctccagggtgaggagaggaaggagagagatggaactagaccggtctgcggggaaacgcgacagttttggtagagggacagtgtcttggtaatgtccagggctccaggaagagatgtccttgtggctggaggcctcaatgcctgatgtttctcctgattctgcaacggagttgtggaagacagaacctcaagatgcaggagaccagggaggcaacacttgcatcctcagggaggaagccaggatgccccagtcaactggggttgctttagggatagggttggagtcagcagagcctacagccctgctccccagggcagagaccctcccagagccgacagagcttcgtccacaaaagcggaaaaagggcccagcccccaaaatgctggggaacgagctgtgcagtgtctgtggggacaaagcctctggcttccattacaacgtgctgagctgcgagggctgcaagggattcttccgccgcagtgtcatcaagggagcacgctatgtctgccacagcggtggccactgccccatggacacctacatgcggcggaaatgccaggagtgtcgacttcgcaaatgccgccaggcaggcatgagggaggagtgtgtgctgtcagaagaacagatccgcttgaagaaactgaagcggcaagaagaggaacaggctcaagccacttcggtgtccccaagggtgtcctcacctcctcaagtcctgccacagctcagcccagagcagctgggcatgatcgagaagctggtggctgcccagcaacagtgtaacaggcgctccttctcagaccgcctgcgcgtcacgccttggcccattgcacccgaccctcagagccgggaagcccgacaacagcgctttgcccactttactgagctggccatcgtgtccgtgcaggagattgttgactttgccaaacagctccctggcttcctacagctcagcagggaggaccagatcgccttgctgaagacctctgcaatcgaggtcatgcttctggagacgtcacggaggtacaaccccggcagtgagagcatcaccttcctcaaggacttcagttacaaccgggaagactttgccaaagcagggctgcaggtggagttcatcaaccccatctttgagttctccagagccatgaatgagctgcaactcaatgatgctgagtttgctctgctcattgccatcagcatcttctctgcagaccggcccaacgtgcaggaccagctccaagtagagaggctgcaacacacatatgtggaggccctgcacgcctacgtctccatcaaccacccccacgaccgactgatgttcccacggatgctaatgaagctggtgagcctccgtactttgagcagcgtccattcagagcaagtgtttgcccttcgcctgcaggacaaaaagcttccccctctgctgtctgagatctgggatgtccacgagtgactgtttcaccgtgtcctttgtgttggccacatggcgaaggctcactgactgcttcccacgggtggagcagactgagaagggcagacattcctgggagctgggtgaaggagagagccttgcgtagcattaagggagagtcaacaggttgggtgttttctggctgctgggcagttgggatctactaacgttgtataccatctgaagaccttgttgacccaaccaaata

SEQ ID NO: 11 - DNA, 625 bp, Mus musculus
gcacaatcaccggtgcggacacagagctctcgcctcccacggccgtttccagggcaacagagtcggagaccccctgcgacccccctcccgatcgccggtgcagtcatgagccccgcctccccctggtgcacggagaggggcggggcctggaacaaggctgcttcgtgacccactatgtcttcccccacaagttctctggacactcccgtgcctgggaatggttctcctcagcccagtacctccgccacgtcacccactattaaggaagaggggcaggagactgatcctcctccaggctctgaagggtccagctctgcctacatcgtggtcatcttagagccagaggatgagcctgagcgcaagcggaagaaggggccggccccgaagatgctgggccatgagctgtgccgcgtgtgcggagacaaggcttcgggcttccactacaacgtgctcagctgtgaaggctgcaaaggcttcttccggcgcagtgtggtccacggtggggccgggcgctatgcctgtcggggcagcggaacctgccagatggatgccttcatgcggcgcaagtgccagctctgccggctgcgcaagtgcaaggaggctggcatgcgggagcagtgcgtgc

SEQ ID NO: 12 - DNA, 362 bp, Mus musculus
tttcgctcagcaagcgctgttgcttcgagctactcccaggcttctgaagttacttccaaagtgctgtggaggcacaatcaccggtgcggacacagagctctcgcctcccacggccgtttccagggcaacagagtcggagaccccctgcgacccccctcccgatcgccggtgcagtcatgagccccgcctccccctggtgcacggagaggggcggggcctggaacaagcaggctgcttcgtgacccactatgtcttcccccacaagttctctggacactcccgtgcctgggaatggttctcctcagcccagtacctccgccacgtcacccactattaaggaagaggggcaggagactgatcct

SEQ ID NO: 13 - DNA, 656 bp, Mus musculus
caagcgctgttgcttcgagctactcccaggcttctgaagttacttccaaagtgctgtggaggcacaatcaccgctctcgcctcccacggccgtttccagggcaacagagtcggagaccccctgcgacccccctcccgatcgccggtgcagtcatgagccccgcctccccctggtgcacggagaggggcggggcctggaacaaggctgcttcgtgacccactatgtcttcccccacaagttctctggacactcccgtgcctgggaatggttctcctcagcccagtacctccgccacgtcacccactattaaggaagaggggcaggagactgatcctcctccaggctctgaagggtccagctctgcctacatcgtggtcatcttagagccagaggatgagcctgagcgcaagcggaagaaggggccggccccgaagatgctgggccatgagctgtgccgcgtgtgcggagacaaggcttcgggcttccactacaacgtgctcagctgtgaaggctgcaaaggcttcttccggcgcagtgtggtccacggtggggccgggcgctatgcctgtcggggcagcggaacctgccagatggatgccttcatgcggcgcaagtgccagctctgccggctgcgcaagtgcaaggaggctggcatg

SEQ ID NO: 14 - DNA, 331 bp, Mus musculus
  misc_feature (316)..(316): n is a, c, g, or t
cttcttgaagatggactcttcacctctcctcctggggtgggaggacattgtcacggcccagtccctcgggctcagcctcaaactcagcggcagttggcactagaaggccccaccccacccattgagtcttccaagagtggtgagggtcacaggtcctagcctctgaccgttcccagctgccctcccacccacgcttacacctcagcctaccacaccatgcaccttgagtggagagaggttagggcaggtggccccccacagttgggagaccacaggccctctcttctgccccttttatttaataaaaaaacaaaantnaaaaaaaaaaaaa

(19) United States
(12) Patent Application Publication
(10) Pub. No.: US 2005/0003998 A1
(43) Pub. Date: Jan. 6, 2005

(54) Therapeutic use of selective LXR modulators

(76) Inventors: Goran Bertilsson, Vasterhaninge (SE); Jessica Heidrich, Arsta (SE); Johan Haggblad, Stockholm (SE); Harriet Ronnholm, Trangsund (SE); Olof Zachrisson, Spanga (SE)

Correspondence Address: Ivor R. Elrifi, Esq.;Mintz, Levin, Cohn, Ferris, Glovsky and Popeo, P.C, The Chrysler Center, 666 Third Avenue, 24th Floor, New York, NY, 10017, US

(21) Appl. No.: 10/641,925

(22) Filed: Aug. 15, 2003

Related U.S. Application Data
(60) Provisional application No. 60/403,618, filed on Aug. 15, 2002.
(60) Provisional application No. 60/431,896, filed on Dec. 9, 2002.

(51) Int. Cl. A61K 31/00

(52) U.S. Cl. 514/1

(57) ABSTRACT

The invention relates generally to methods of influencing central nervous system cells to produce progeny useful in the treatment of CNS disorders. More specifically, the invention includes methods of exposing a patient suffering from such a disorder to a reagent that modulates the proliferation, migration, differentiation and survival of central nervous system cells via LXR signaling. These methods are useful for reducing at least one symptom of the disorder.

### RELATED APPLICATIONS

This application claims the benefit of priority from U.S. Ser. No. 60/407,863 filed Aug. 15, 2002. The contents of this applications are incorporated herein by reference in their entirety.

### FIELD OF THE INVENTION

The invention relates generally to methods of influencing adult neural stem cells and neural progenitor cells to produce progeny that can replace damaged or missing neurons or other central nervous system (CNS) cell types. More specifically, the invention includes methods of exposing a patient suffering from a disorder to a reagent that regulates the differentiation, proliferation, survival and migration of central nervous system cells via modulation of liver X-receptor (LXR) signaling. These methods are useful for reducing at least one symptom of a CNS disorder.

### BACKGROUND OF THE INVENTION

Throughout this specification, various patents, published patent applications and scientific references are cited to describe the state and content of the art. Those disclosures, in their entireties, are hereby incorporated into the present specification by reference.

For several years, it has been known that neural stem cells exist in the adult mammalian brain. This concept is of particular importance since the adult brain was thought to have very limited regenerative capacity. Moreover, the possibility to use adult-derived stem cells for tissue repair may help to overcome the ethical problems associated with embryonic cell research. Although the generation of neurons and glia can be observed in the adult brain, there is thus far only limited knowledge about stimulation of human neural stem cells in vitro and in vivo.

The first suggestions that new neurons were born in the adult mammalian brain came from studies performed in the 1960s (Altman, J. and G. Das (1965). J Comp Neurol 124: 319-335. Altman, J. and G. Das (1967) Nature 214: 1098-1101). It however took another three decades and refined technical procedures to overthrow the dogma that neurogenesis within the mammalian CNS is restricted to embryogenesis and the perinatal period (for review see Momma, S., C. B. Johansson, et al. (2000). Curr Opin Neurobiol 10(1): 45-9; Kuhn, H. G. and C. N. Svendsen (1999) Bioessays 21(8): 625-30). Treatment of neural disease and injury traditionally focuses on keeping existing neurons alive, but possibilities now arise for exploiting neurogenesis for therapeutic treatments of neurological disorders and diseases.

The source of new neurons is neural stem cells (NSC), located within the ependymal and/or subventricular zone (SVZ) lining the lateral ventricle (Doetsch, F., I. Caille, et al. (1999) Cell 97(6): 703-16; Johansson, C. B., S. Momma, et al. (1999) Cell 96(1): 25-34) and in the dentate gyrus of the hippocampus formation (Gage, F. H., G. Kempermann, et al. (1998) J Neurobiol 36(2): 249-66). Recent studies reveal the potential for several additional locations of NSC within the adult CNS (Palmer, T. D., E. A. Markakis, et al. (1999) J Neurosci 19(19): 8487-97). Asymmetric division of NSC maintain their number while generating a population of rapidly dividing precursor or progenitor cells (Johansson, C. B., S. Momma, et al. (1999) Cell 96(1): 25-34). The progenitors respond to a range of cues that dictate the extent of their proliferation and their fate, both in terms of the cell type that they differentiate into and the position that they ultimately take up in the brain.

The NSC of the ventricular system in the adult are likely counterparts of the embryonic ventricular zone stem cells lining the neural tube whose progeny migrate away to form the CNS as differentiated neurons and glia (Jacobson, M. (1991) Developmental Neurobiology, Plenum Press, New York: 401-451). NSC persist in the adult lateral ventricle wall (LVW), generating neuronal progenitors which migrate down the rostral migratory stream to the olfactory bulb, where they differentiate into granule cells and periglomerular neurons (Lois, C. and A. Alvarez-Buylla (1993) Proc Natl Acad Sci USA 90(5): 2074-7). Substantial neuronal death occurs in the olfactory bulb generating a need for continuous replacement of lost neurons, a need satisfied by the migrating progenitors derived from the LVW (Biebl, M., C. M. Cooper, et al. (2000) Neurosci Lett 291(1): 17-20). Further to this ongoing repopulation of olfactory bulb neurons, there are forceful indications that lost neurons from other brain regions can be replaced by progenitors from the LVW that differentiate into the lost neuron phenotype complete with appropriate neuronal projections and synapses with the correct target cell type (Snyder, E. Y., C. Yoon, et al. (1997) Proc Natl Acad Sci USA 94(21): 11663-8; Magavi, S. S., B. R. Leavitt, et al. (2000) Nature 405(6789): 951-5).

In vitro cultivation techniques have been established to identify the external signals involved in the regulation of NSC proliferation and differentiation (Johansson, C. B., S. Momma, et al. (1999) Cell 96(1): 25-34; Johansson, C. B., M. Svensson, et al. (1999). Exp Cell Res 253(2): 733-6). The mitogens EGF and basic FGF allow neural progenitors, isolated from the ventricle wall and hippocampus, to be greatly expanded in culture (McKay, R. (1997) Science 276(5309): 66-71; Johansson, C. B., M. Svensson, et al. (1999). Exp Cell Res 253(2): 733-6). The dividing progenitors remain in an undifferentiated state growing into large balls of cells known as neurospheres. Withdrawal of the mitogens combined with addition of serum induces differentiation of the progenitors into the three cell lineages of the brain: neurons, astrocytes and oligodendrocytes (Doetsch, F., 1. Caille, et al. (1999) Cell 97(6): 703-16; Johansson, C. B., M. Svensson, et al. (1999). Exp Cell Res 253(2): 733-6). Application of specific growth factors can distort the proportions of each cell type in one way or another. For example, CNTF acts to direct the neural progenitors to an astrocytic fate (Johe, K. K., T. G. Hazel, et al. (1996) Genes Dev 10(24): 3129-40), while the thyroid hormone, triiodothyronine (T3) has been shown to promote oligodendrocyte differentiation (Johe, K. K., T. G. Hazel, et al. (1996) Genes Dev 10(24): 3129-40). Enhancement of neuronal differentiation of neural progenitors by PDGF has also been documented (Johe, K. K., T. G. Hazel, et al. (1996) Genes Dev 10(24): 3129-40; Williams, B. P., J. K. Park, et al. (1997) Neuron 18(4): 553-62).

The ability to expand neural progenitor cells and then manipulate their cell fate has also had enormous implications in transplant therapies for neurological diseases in which specific cell types are lost. The most obvious example is Parkinson's Disease (PD) which is characterized by degeneration of dopaminergic neurons in the substantia nigra. Previous transplantation treatments for PD patients have used fetal tissue taken from the ventral midbrain at a time when substantia nigral dopaminergic neurons are undergoing terminal differentiation (Herman, J. P. and N. D. Abrous (1994) Prog Neurobiol 44(1): 1-35). The cells are grafted onto the striatum where they form synaptic contacts with host striatal neurons, their normal synaptic target, restoring dopamine turnover and release to normal levels with significant functional benefits to the patient (Herman, J. P. and N. D. Abrous (1994) Prog Neurobiol 44(1): 1-35) (for review see (Bjorklund, A. and O. Lindvall (2000) Nat Neurosci 3(6): 537-44)). Grafting of fetal tissue is hindered by lack of donor tissue. In vitro expansion and manipulation of NSC, however, can potentially provide a range of well characterized cells for transplant-based strategies for neurodegenerative diseases, such as dopaminergic cells for PD. To this aim, the identification of factors and pathways that govern the proliferation and differentiation of neural cell types will prove fundamental.

Ultimately the identification of these proliferative and differentiating factors is likely to provide insights into the stimulation of endogenous neurogenesis for the treatment of neurological diseases and disorders. Intraventricular infusion of both EGF and basic FGF have been shown to proliferate the ventricle wall cell population, and in the case of EGF, extensive migration of progenitors into the neighboring striatal parenchyma (Craig, C. G., V. Tropepe, et al. (1996) J Neurosci 16(8): 2649-58; Kuhn, H. G., J. Winkler, et al. (1997) J Neurosci 17(15): 5820-9). The progenitors differentiated predominantly into a glial lineage while reducing the generation of neurons (Kuhn, H. G., J. Winkler, et al. (1997) J Neurosci 17(15): 5820-9). A recent study found that intraventricular infusion of BDNF in adult rats stimulates an increase in the number of newly generated neurons in the olfactory bulb and rostral migratory stream, and in parenchymal structures, including the striatum, septum, thalamus and hypothalamus (Pencea, V., K. D. Bingaman, et al. (2001) J Neurosci 21(17): 6706-17). These studies demonstrate that the proliferation of progenitors within the SVZ of the LVW can be stimulated and that their lineage can be manipulated to neuronal and glial fates. Currently the number of factors known to affect neurogenesis in vivo is small and their effects are either undesired or limited.

Therefore, there is a long felt need to identify other factors that can selectively stimulate neural stem cell activity through proliferation of neural progenitors and differentiation into the desired neuronal cell type. This activity would be beneficial for both stimulation of in vivo neurogenesis and culture of cells for transplantation therapy. The present invention demonstrates a role for LXR modulators and their effects on the proliferation, differentiation, survival and migration of neural stem cells in vitro and in vivo.

### SUMMARY OF THE INVENTION

This invention relates generally to methods of influencing central nervous system cells to produce progeny that can replace damaged or missing neurons or other central nervous system cell types. The role of lipids and the effects of perturbation of lipid homeostasis in relation to neurogenesis and its role in alleviation of symptoms of psychiatric and neurological disorders is an emerging field of research where this invention is pioneering a new field of therapeutic opportunities for modulators of LXR.

As used here, LXR modulators are agents that modulate the activity of the LXR subtypes directly through interaction with the LXR or indirectly by interaction with a heterodimerization partner like e.g. the retinoic X-receptors (RXRs) (Edwards PA (2002) Vascul Pharmacol. 38(4):249-56). In nuclear receptor signaling the interaction of a nuclear receptor homo- or heterodimer with the transcription complex gives rise to the final agonistic or antagonistic response. A given agent can be agonistic in one tissue and antagonistic in another tissue depending on the downstream signaling entities present in the cells of the tissue. As an example, tamoxifen is agonistic in bone tissue while being antagonistic to estrogen action in breast tumor tissue, i.e. any given nuclear receptor modulator has its own response profile in different tissues (e.g. U.S. Pat. Nos. 6,576,645, 6,528,681, 6,436,923).

In one aspect, the invention includes a method of alleviating a symptom of a disorder of the nervous system comprising administering a selective LXR modulator (SLRM) to modulate neural stem cell or neural progenitor cell activity in vivo to a patient suffering from the disease or disorder of the nervous system. For the purposes of this disclosure, disorder and disease shall have the same meaning.

In another aspect, the invention provides a method of modulating an LXR or an RXR or a combination thereof, in a neural stem cell or neural progenitor cell, the method comprising exposing the cell expressing the receptor to exogenous reagent, antibody, or affibody, wherein the exposure induces or inhibits the neural stem cell or neural progenitor cell to proliferate, differentiate or survive.

In a further aspect, the invention includes a method for reducing a symptom of a disease or disorder of the central nervous system in a mammal in need of such treatment comprising administering e.g., but not limited to, oxysterols, TO-90137 or other LXR activators and SLRMs, 9-cis retinoic acid, cis-4,7,10,13,16,19-docosahexaeoic acid or other RXR activators to the mammal.

In another aspect, the invention provides a method for inducing the in situ proliferation, migration, differentiation or survival of a neural stem cell or neural progenitor cell located in the neural tissue of a mammal, the method comprising administering a therapeutically effective amount of e.g., but not limited to, oxysterols, TO-90137 or other LXR activators and SLRMs, 9-cis retinoic acid, cis-4,7,10,13,16,19-docosahexaeoic acid or other RXR activators to the neural tissue to modulate the proliferation, migration differentiation or survival of the cell.

In another aspect, the invention includes a method of enhancing neurogenesis in a patient suffering from a disease or disorder of the central nervous system, by infusion, enteral-, transdermal-, buccal-, nasal- or other administration of e.g., but not limited to, oxysterols, TO-90137 or other LXR activators and SLRMs, 9-cis retinoic acid, cis-4,7,10,13,16,19-docosahexaeoic acid or other RXR activators.

In another aspect, the invention provides a method for producing a population of cells enriched for human neural stem cells or human neural progenitor cells, comprising: (a) contacting a population containing neural stem cells or neural progenitor cells with a reagent that recognizes a determinant on an LXR or RXR; and (b) selecting for cells in which there is contact between the reagent and the determinant of the cells of step (a), to produce a population highly enriched for central nervous system stem cells. In one embodiment of the invention, the reagent is selected from the group consisting of a soluble receptor, a small molecule, a peptide, an antibody and an affibody. In another embodiment of the invention, the soluble receptor is an LXR or an RXR.

In a further aspect, the invention includes an in vitro cell culture comprising a cell population generated by the method previously described wherein the cell population is enriched in receptor expressing cells wherein the receptors are selected from the group consisting of LXRs and RXRs.

In one aspect, the invention includes a method for alleviating a symptom of a disease or disorder of the central nervous system comprising administering the population of cells described above to a mammal in need thereof. In a further aspect, the invention includes a non-human mammal engrafted with the human neural stem cells or neural progenitor cells previously described. In a preferred embodiment of the invention, the non-human mammal is selected from the group including rat, mouse, rabbit, horse, sheep, pig, guinea pig and primate.

In another aspect, the invention includes a method of reducing a symptom of a disease or disorder of the central nervous system in a subject comprising the steps of administering into the spinal cord of the subject a composition comprising a population of isolated neural stem cells or neural progenitor cells obtained from fetal or adult tissue; and e.g., but not limited to, oxysterols, TO-90137 or other LXR activators and SLRMs, 9-cis retinoic acid, cis-4,7,10,13,16,19-docosahexaeoic acid or other RXR activators or a combination thereof such that the symptom is reduced.

In another aspect, the invention includes a method of gene delivery and expression in a target cell of a mammal comprising the steps of: (a) providing an isolated nucleic acid fragment of a nucleic acid sequence which encodes for LXRs or RXRs; (b) selecting a viral vector with at least one insertion site for insertion of the isolated nucleic acid fragment operably linked to a promoter capable of expression in the target cells; (c) inserting the isolated nucleic acid fragment into the insertion site, and (d) introducing the vector into the target cell wherein the gene is expressed at detectable levels. In one embodiment of the invention, the virus is selected from the group consisting of retrovirus, adenovirus, pox virus, iridoviruses, coronaviruses, togaviruses, caliciviruses, lentiviruses, adeno-associated viruses and picomaviruses. In another embodiment of the invention, the pox virus is vaccinia. In another embodiment of the invention, the virus is a strain that has been genetically modified or selected to be non-virulent in a host.

In a further aspect, the invention includes a method for alleviating a symptom of a disease or disorder of the central nervous system in a patient comprising the steps of: (a) providing a population of neural stem cells or neural progenitor cells; (b) suspending the neural stem cells or neural progenitor cells in a solution comprising a mixture comprising e.g., but not limited to, oxysterols, TO-90137 or other LXR activators and SLRMs, 9-cis retinoic acid, cis-4,7,10,13,16,19-docosahexaeoic acid or other RXR activators to generate a cell suspension; (c) delivering the cell suspension to an injection site in the central nervous system of the patient to alleviate the symptom. In one embodiment of the invention, the method described further comprises the step of injecting the injection site with a growth factor for a period of time before the step of delivering the cell suspension. In another embodiment of the invention, the method described further comprises the step of injecting the injection site with the factors after the delivering step.

In a further aspect, the invention includes a method for transplanting a population of cells enriched for human neural stem cells or human neural progenitor cells, comprising: (a) contacting a population containing neural stem cells or neural progenitor cells with a reagent that recognizes a determinant on an LXR or an RXR; (b) selecting for cells in which there is contacted between the reagent and the determinant of the cells of step (a), to produce a population highly enriched for central nervous system stem cells; and (c) implanting the selected cells of step (b) into a non-human mammal.

In a further aspect, the invention includes a method of modulating LXR and RXR expression in a neural stem cell or neural progenitor cell comprising the step of exposing the cell expressing the receptor, or ligand to exogenous reagent, antibody, or affibody, wherein the exposure induces the neural stem cell or neural progenitor cell to proliferation, differentiation or survival. In one embodiment of the invention the neural stem cell or neural progenitor cell is derived from fetal brain, adult brain, neural cell culture or a neurosphere. In a further aspect, the invention includes a method of determining an isolated candidate LXR or RXR modulator compound for its ability to modulate neural stem cell or neural progenitor cell activity comprising the steps of: (a) administering the isolated candidate compound to a non-human mammal; and (b) determining if the candidate compound has an effect on modulating the neural stem cell or neural progenitor cell activity in the non-human mammal. In one embodiment of the invention, the determining step comprises comparing the neurological effects of the non-human mammal with a referenced non-human mammal not administered the candidate compound. In a further embodiment of the invention, the compound is selected from the group consisting of a peptide, a small molecule, and a receptor agonist. The neural stem cell or neural progenitor cell activity could be proliferation, differentiation, migration or survival.

All references, patent applications, and patents cited in this specification, for any reason, are hereby incorporated by reference in their entireties.

### BRIEF DESCRIPTION OF DRAWINGS

FIG. 1 depicts RT-PCR results of presence of LXRs in cultured mouse stem cells.

FIG. 2 depicts western blot results of presence of LXRs in cultured mouse stem cells.

FIG. 3 depicts effect of in vitro proliferation of neurospheres exerted by; a/LXR- and RXR agonists b/synthetic LXR agonist TO-901317.

FIG. 4 depicts effects of oral administration of LXR agonist TO-901317 in the lateral ventricle wall.

FIG. 5 depicts effects of intracerebroventricular administration of LXR agonist TO-901317 in the lateral ventricle wall.

### DETAILED DESCRIPTION OF THE INVENTION

It has been discovered that certain reagents are capable of modulating the differentiation, migration, proliferation and survival of neural stem/progenitor cells both in vitro and in vivo. As used herein, the term “modulate” refers to having an affect in such a way as to alter the differentiation, migration, proliferation and survival of neural stem cell (NSC) or neural progenitor cell (NPC) activity. Since undifferentiated, pluripotent stem cells can proliferate in culture for a year or more, the invention described in this disclosure provides an almost limitless supply of neural precursors.

Throughout this disclosure, the term “neural stem cell” (NSC) includes “neural progenitor cell,” “neuronal progenitor cell,” “neural precursor cell,” and “neuronal precursor cell” (all referred to herein as NPC). NSC and NPC encompasses both a single cell or a plurality of cell (e.g., a cell population). These cells can be identified by their ability to undergo continuous cellular proliferation, to regenerate exact copies of themselves (self-renew), to generate a large number of regional cellular progeny, and to elaborate new cells in response to injury or disease. The term NPCs mean cells that can generate progeny that are either neuronal cells (such as neuronal precursors or mature neurons) or glial cells (such as glial precursors, mature astrocytes, or mature oligodendrocytes). Typically, the cells express some of the phenotypic markers that are characteristic of the neural lineage. They also do not usually produce progeny of other embryonic germ layers when cultured by themselves in vitro unless dedifferentiated or reprogrammed in some fashion. As used herein, the term “neurosphere” refers to the ball of cells consisting of NSC.

As used herein, the term “reagent” refers to any substance that is chemically and biologically capable of activating a receptor, including peptides, small molecules, antibodies (or fragments thereof), affibodies and any molecule that dimerizes or multimerizes the receptors or replaces the need for activation of the extracellular domains. In one embodiment, the reagent is a small molecule.

As used herein, the term “antibody” or “immunoglobulin” as used in this disclosure refers to both polyclonal and monoclonal antibody and functional derivatives (i.e., engineered antibody) thereof. Antibodies can be whole immunoglobulin of any class, e.g., IgG, IgM, IgA, IgD, IgE, or hybrid antibodies with dual or multiple antigen or epitope specificities, or fragments, e.g., F(ab′)2, F(ab)2, Fab′, Fab1 and the like, including hybrid fragments. Functional derivatives include engineered antibodies. The ambit of the term deliberately encompasses not only intact immunoglobulin molecules, but also such fragments and derivatives of immunoglobulin molecules (such as single chain Fv constructs, diabodies and fusion constructs) as may be prepared by techniques known in the art, and retaining a desired antibody binding specificity. The term “affibody” (U.S. Pat. No. 5,831,012) refers to highly specific affinity proteins that can be designed to bind to any desired target molecule. These antibody mimics can be manufactured to have the desired properties (specificity and affinity), while also being highly robust to withstand a broad range of analytical conditions, including pH and elevated temperature. The specific binding properties that can be engineered into each capture protein allow it to have very high specificity and the desired affinity for a corresponding target protein. A specific target protein will thus bind only to its corresponding capture protein. The small size (only 58 amino acids), high solubility, ease of further engineering into multifunctional constructs, excellent folding and absence of cysteines, as well as a stable scaffold that can be produced in large quantities using low cost bacterial expression systems, make affibodies superior capture molecules to antibodies or antibody fragments, such as Fab or single chain Fv (scFv) fragments, in a variety of Life Science applications. The term antibodies also encompasses engineered antibodies.

As used herein, the term “engineered antibody” encompasses all biochemically or recombinantly produced functional derivatives of antibodies. A protein is a functional derivative of an antibody if it has at least one antigen binding site (ABS) or a complementarity-determining region (CDR) that when combined with other CDR regions (on the same polypeptide chain or on a different polypeptide chain) can form an ABS. The definition of engineered antibody would include, at least, recombinant antibodies, tagged antibodies, labeled antibodies, Fv fragments, Fab fragments, recombinant (as opposed to natural) multimeric antibodies, single chain antibodies, diabodies, triabodies, tetravalent multimers (dimer of diabodies), pentavalent multimers (dimer of diabody and triabody), hexavalent multimers (dimer of triabodies) and other higher multimeric forms of antibodies.

The terms “recombinant nucleic acid” or “recombinantly produced nucleic acid” refer to nucleic acids such as DNA or RNA which has been isolated from its native or endogenous source and modified either chemically or enzymatically by adding, deleting or altering naturally-occurring flanking or internal nucleotides. Flanking nucleotides are those nucleotides which are either upstream or downstream from the described sequence or sub-sequence of nucleotides, while internal nucleotides are those nucleotides which occur within the described sequence or subsequence.

The term “recombinant means” refers to techniques where proteins are isolated, the cDNA sequence coding the protein identified and inserted into an expression vector. The vector is then introduced into a cell and the cell expresses the protein. Recombinant means also encompasses the ligation of coding or promoter DNA from different sources into one vector for expression of a PPC, constitutive expression of a protein, or inducible expression of a protein.

The term “promoter” refers to a DNA sequence which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent if the promoter is a constitutive promoter.

The term “enhancer” refers to a promoter element. An enhancer can increase the efficiency with which a particular gene is transcribed into mRNA irrespective of the distance or orientation of the enhancer relative to the start site of transcription.

“Complementary DNA (cDNA)” refers to a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complement. “Expression” refers to the process by which a polypeptide is produced from a structural gene. The process involves transcription of the gene into mRNA and the translation of such mRNA into polypeptide(s). “Cloning vector” refers to a DNA molecule, such as a plasmid, cosmid, phagemid, or bacteriophage, which has the capability of replicating autonomously in a host cell and which is used to transform cells for gene manipulation. Cloning vectors typically contain one or a small number of restriction endonuclease recognition sites at which foreign DNA sequences may be inserted in a determinable fashion without loss of an essential biological function of the vector, as well as a marker gene which is suitable for use in the identification and selection of cells transformed with the cloning vector. Marker genes typically include genes that provide tetracycline resistance or ampicillin resistance. “Expression vector” refers to a DNA molecule comprising a cloned structural gene encoding a foreign protein which provides the expression of the foreign protein in a recombinant host. Typically, the expression of the cloned gene is placed under the control of (i.e., operably linked to) certain regulatory sequences such as promoter and enhancer sequences. Promoter sequences may be either constitutive or inducible.

“Recombinant Host” refers to a prokaryotic or eukaryotic cell which contains either a cloning vector or expression vector. This term is also meant to include those prokaryotic or eukaryotic cells that have been genetically engineered to contain the cloned gene(s) in the chromosome or genome of the host cell. The host cell is not limited to a unicellular organism. Multicellular organisms such as mammals, insects, and plants are also contemplated as host cells in the context of this invention. For examples of suitable hosts, see Sambrook et al., MOLECULAR CLONING: A LABORATORY MANUAL, Second Edition, Cold Spring Harbor Laboratory, Cold Spring Harbor, N.Y. (1989).

The term “treating” in its various grammatical forms in relation to the present invention refers to preventing, curing, reversing, attenuating, alleviating, minimizing, suppressing or halting the deleterious effects of a disease state, disease progression, disease causative agent (e.g., bacteria or viruses) or other abnormal condition.

The terms “recombinant protein,” “recombinantly produced protein” refer to a peptide or protein produced using non-native cells that do not have an endogenous copy of DNA able to express the protein. The cells produce the protein because they have been genetically altered by the introduction of the appropriate nucleic acid sequence. The recombinant protein will not be found in association with proteins and other subcellular components normally associated with the cells producing the protein.

According to the specific case, the “therapeutically effective amount” of an agent should be determined as being the amount sufficient to improve the symptoms of the patient in need of treatment or at least to partially arrest the disease and its complications. Amounts effective for such use will depend on the severity of the disease and the general state of the patient's health. Single or multiple administrations may be required depending on the dosage and frequency as required and tolerated by the patient.

The terms “binding specificity,” “specifically binds to” or “specifically immunoreactive with,” when referring to a protein, antibody, or antibody binding site (ABS) of the invention, refers to a binding reaction which is determinative of the presence of the protein or carbohydrate in the presence of a heterogeneous population of proteins and other biologics. A variety of immunoassay formats may be used to determine binding. For example, solid-phase ELISA immunoassays are routinely used to select antibodies specifically immunoreactive with a protein or carbohydrate. See Harlow & Lane, Antibodies, A Laboratory Manual, Cold Spring Harbor Publication, New York (1988) for a description of immunoassay formats and conditions that can be used to determine specific immunoreactivity.

The terms “nucleic acid encoding” or “nucleic acid sequence encoding” refer to a nucleic acid which directs the expression of a specific protein or peptide. The nucleic acid sequences include both the DNA strand sequence that is transcribed into RNA and the RNA sequence that is translated into protein. The nucleic acid sequences include both full length nucleic acid sequences as well as shorter sequences derived from the full length sequences. It is understood that a particular nucleic acid sequence includes the degenerate codons of the native sequence or sequences which may be introduced to provide codon preference in a specific host cell. The nucleic acid includes both the sense and antisense strands as either individual single strands or in the duplex form.

“Pharmaceutical composition” refers to formulations of various preparations. Parenteral formulations are known and are preferred for use in the invention. The formulations containing therapeutically effective amounts of the pharmaceutical agents are either sterile liquid solutions, liquid suspensions or lyophilized versions and optionally contain stabilizers or excipients. Lyophilized compositions are reconstituted with suitable diluents, e.g., water for injection, saline, 0.3% glycine and the like, at a level of about from 0.01 mg/kg of host body weight to 10 mg/kg or more.

Preferred reagents of the invention, are LXR activators and/or SLRMs and include members of the oxysterols (e.g. 22-hydroxy- 24-hydroxy- and 25-hydroxysterols), 5-Cholesten-24(s), 25-epoxy-3b-ol, GW3965, XCT0179628, TO-90137, 5-tetradecyloxy-2-furancarboxylic acid (TOFA), paxilline, or other LXR activators and SLRMs described e.g. in U.S. Pat. Nos. 6,545,049; 6,316,503; 6,184,215; 6,071,955; in PCT/US02/28147 and WO0224632: and 9-cis retinoic acid, cis-4,7,10,13,16,19-docosahexaeoic acid or other RXR activators—or prodrugs of aforementioned preferred reagents.

The invention provides a method for in vivo modulation of LXR activity, and for therapeutic administration of reagents like e.g., but not limited to, oxysterols, TO-90137 or other LXR activators and SLRMs, 9-cis retinoic acid, cis-4,7,10,13,16,19-docosahexaeoic acid or other RXR activators for drug screening. In one embodiment, the compounds above described are administered to neural tissue. In a preferred embodiment, the neural tissue is fetal or adult brain. In yet another embodiment, the population containing neural or neural-derived cells is obtained from a neural cell culture or neurosphere.

### LXR Receptors, Ligands and Lipid Homeostasis

The liver X receptors alpha and beta (LXRalpha or NR1H3 and LXRbeta or NR1H2) are members of the nuclear receptor family of proteins and play a key role for the control of cholesterol metabolism and transport, glucose metabolism and inflammation (Repa, J. J. and Mangelsdorf, D. J., (2002) Nat Med 8(11):1243-1248; Cao, G. et al, (2003) J Biol Chem 278(2):1131-1136; Joseph, S. B. et al, (2003) Nat Med 9(2):213-219). LXRs form heterodimeric complexes with the retinoic acid receptors. The two known LXR isoforms, alpha and beta, are differentially expressed; the alpha isoform is predominantly expressed in liver, intestine, kidney, spleen, brown and white adipose tissue and lung whereas the beta isoform expression has been shown to be less restricted. There is a high degree of conservation of the LXRs between rodents and man.

The endogenous activators of the LXRs are oxysterols and intermediates in the cholesterol biosynthetic pathway (Janowski, B. A. el al, (1996) Nature 383(6602):728-31; Lehmann, J. M. el al, (1997) J Biol Chem 272(6):3137-40). The receptors regulate mechanisms of cholesterol synthesis, dietary cholesterol absorption, reverse cholesterol transport and bile acid and synthesis and -excretion. In particular the effects of LXR activation have been studied for the ATP-binding cassette serol transporter ABCA 1, ABCG 1, ABCG5 and ABCG8 (Laffitte, B. A. et al (2001). Proc Natl Acad Sci USA 98(2): 507-12; Costet, P. et al (2000). J Biol Chem 275(36):28240-5; Repa, J. J. el al, (2000) Science 289(5484):1524-9; Venkateswaran, A. et al, (2000) Proc Natl Acad Sci USA 97(22):12097-102; Schwartz, K. et al, (2000) Biochem Biophys Res Commun 274(3):794-802; Repa, J. J. et al, (2002) J Biol Chem 277(21):18793-18800). Moreover, LXR regulate key genes in lipid metabolism including apoE, apoCIII, LPL, PLTP and CETP (Luo, Y. and Tall, A. R., (2000) J Clin Invest 105(4): 513-520; Zhang, Y. et al, (2001) J Biol Chem 276(46): 43018-43024; Cao, G. et al, (2002) J Biol Chem 277(42):39561-39565; Mak, P. A. et al, (2002) J Biol Chem 277(35):31900-31908). LXR activation has also been reported to increase coordinate expression of major fatty-acid biosynthetic genes and to increase plasma triglyceride and phospholipid levels. This effect is thought to be mediated through the SREBP-1 lipogenic program (Schultz et al, Genes Dev 2000 14 (22):2831-2838). LXR gene regulation is proposed to be anti-atherogenic, which has been proven in mouse models (Joseph, S. B. et al, (2002) Proc Natl Acad Sci USA 99(11):7604-7609; Tangirala, R. K. et al, (2002) Proc Natl Acad Sci USA 99(18): 11896-11901). Synthetic LXR agonists inhibit the development of atherosclerosis in murine models, an effect that is likely to result from the modulation of both metabolic and inflammatory gene expression. These observations identify the LXR pathway as a potential target for therapeutic intervention in human cardiovascular disease and indeed many pharmaceutical efforts are directed to this goal.

Interestingly, proliferative PPARα ligands can suppress LXR mediated transcription in COS-7 cells and suppress de novo sterol synthesis in rat hepatoma cell line H4IIEC3 (Johnson, T. E. and Ledwith, B. J., (2001) J Steroid Biochem Mol Biol 77(1):59-71.

### Direct and Indirect Actions by LXR Activation

LXRs act as sterol sensors in the mevalonate pathway. Products of mevalonate metabolism are critical for several cellular processes of eukaryotic cells, and inhibition of the mevalonate pathway has pleiotrophic effects (Bocan, T. M. (2002) Curr Opin Investig Drugs (9):1312-7). For instance, proliferation of cells require at least two products synthesised from mevalonate; cholesterol and nonsterol isoprenoid derivatives like farnesylated proteins (Goldstein J L, (1990) 343;425-430; Gueddari-Pouzols N et al (2001) J Biomed & Biotech 1(3);108-113). Given that LXR activation can increase the local availability of cholesterol through transport mechanisms described above, it may be argued that it would promote or facilitate proliferative events.

However, in a non-hepatic system, activators of LXRs stimulate epidermal differentiation and development and inhibit keratinocyte proliferation. The LXR agonists 22(R)-hydroxy-cholesterol and 25-hydroxycholesterol, and a nonsterol activator GW3965 were able to reduce irritant dermatitis induced by applying phorbol 12-myristate-13-acetate to the surface of the ears of CD1 mice. LXR agonists displayed a potent anti-inflammatory effect in both irritant and allergic contact models of dermatitis, which required the participation of both LXRα and LXRβ (Fowler, A. J. et al (2003) J Invest Dermatol 120(2):246-55). In contrast to the above, a recent study suggest that phospholipase A2 (PLA2) activity is upregulated through an LXR dependent mechanism in rat smooth muscle cells (Antonio V et al (2003) Biochem J. , ahead of print Jul 28, Pubmed ID no: 12882648). The authors observed that oxysterol ligands that bind to the LXR, including 25-hydroxycholesterol and 22(R)-hydroxycholesterol, cause the accumulation of PLA2 mRNA and an increased enzymatic activity. Transient transfection experiments demonstrated that the PLA2 promoter is synergistically activated by 22(R)-hydroxycholesterol in combination with 9-cis-retinoic acid, a ligand for the LXR heterodimeric partner RXR. Promoter activity was also increased in a sterol-responsive fashion when cells were cotransfected with LXRalpha/RXRalpha or LXRbeta/RXRalpha. These results are indicative of an proinflammatory proliferative action of LXR activation.

Thus, it is not known whether LXR activation is either mediating differentiation or proliferation. This invention proposes that the actions of LXR activation are two-fold; on the one hand indirectly by locally providing lipid metabolites required for cell growth; and secondly a more direct action by regulating transcriptional events involved in the cell cycle. This multitude of responses can be tuned by the use of chemical agents, e.g. SLRMs, triggering the desired tissue response including actions on resident stem and progenitor cells.

### LXRs in the Central Nervous System

Information regarding LXR action in the central nervous system (CNS) is considerably less abundant as compared to information regarding lipid metabolism in the liver. Both LXR subtypes are expressed in the brain, LXRbeta more abundantly, and their roles in this tissue remain largely unexplored and unknown (Whitney, K. D. et al (2002) Mol Endocrinol 16(6):1378-85). LXR agonists have marked effects on gene expression in murine brain tissue both in vitro and in vivo. In primary astrocyte cultures, LXR agonists regulated several established LXR target genes, including ABCA1, and enhanced cholesterol efflux. In contrast, little or no effect on gene expression or cholesterol efflux was detected in primary neuronal cultures. Treatment of mice with LXR agonists resulted in the induction of several LXR target genes related to cholesterol homeostasis in the cerebellum and hippocampus suggesting that LXRs also regulate cholesterol homeostasis in the central nervous system (Whitney, K. D. et al (2002) Mol Endocrinol 16(6):1378-85). Dysregulation of cholesterol balance is implicated in central nervous system diseases such as Alzheimer's and Niemann-Pick disease, thus pharmacological manipulation of the LXRs may prove beneficial in the treatment of these disorders.

In adult rat brain there is high expression of ABCA1 in neurons in the hypothalamus, thalamus, amygdala, cholinergic basal forebrain, and hippocampus (Koldamova et al, J Biol Chem (2003) 278(15):13244-56). The basal ABCA1 mRNA and protein levels detected in these cell types were increased markedly after exposure to oxysterols and 9-cis-retinoic acid, which are ligands for the LXRs and retinoic X receptors (RXR), respectively. Furthermore, these ligands alone or in combination with apoA-I caused a substantial reduction in the stability of amyloid precursor protein C-terminal fragments and decreased amyloid beta production. In another study the induction of the cholesterol transporter ABCA1 in CNS cells by LXR agonists increased secreted beta amyloid levels. The ABCA1 message was also upregulated in neurons and glia in areas of damage by hippocampal AMPA lesion after 3-7 days (Fukumoto et al, (2002) J Biol Chem 277(50):48508-13) pointing to involvement of the lipid transporter system as a damage response.

The effects of a double knock-out of the LXRs have been studied in the CNS and several severe abnormalities were found (Wang et al (2002) Proc Natl Acad Sci USA 99(21):13878-83). One of the most striking features was that the lateral ventricles were closed and lined with lipid-laden cells. In addition, there were enlarged brain blood vessels, especially in the pars reticularis of the substantia nigra and in the globus pallidus. Other features of the brains were excessive lipid deposits, proliferation of astrocytes, loss of neurons, and disorganized myelin sheaths. Electron micrographs revealed that, as mice aged, lipid vacuoles accumulated in astrocytes surrounding blood vessels. Comparison of mRNA profiles in LXR knockout mice and wild-type littermates showed that expression of several LXR target genes involved in cholesterol efflux from astrocytes was reduced. These findings suggest that LXRs have an important function in lipid homeostasis in the brain, and that loss of these receptors results in neurodegenerative processes.

In vitro, treatment of rat pheochromocytoma cells with the LXR agonist 5-tetradecyloxy-2-furancarboxylic acid (TOFA) and the natural LXR agonist, 22(R)-hydroxycholesterol induces neuronal differentiation measured as neurite outgrowth (Schmidt et al, (1999) Mol Cell Endocrinol 155(1-2):51-60). Other papers describe protective action of e.g. LXR ligand 22R-hydroxycholesterol and prevention of beta-amyloid induced apoptosis in Alzheimer's disease (Yao et al, (2002) J Neurochem 83(5):1110-9). These findings suggest that cholesterol/phospholipid metabolism and -transport somehow are involved in neural commitment processes of cultured cells and that an LXR agonist can influence pathological mechanisms with a degree of specificity.

One of the characteristic events in Alzheimer's disease (AD) is the accumulation of amyloid. Amyloid consists of protein fragments and its production and break down by cells is normal. However, in AD, fragments of amyloid called beta-amyloid accumulate to form plaques. Over time, the plaques cause neurons to malfunction and die. Published data suggest that elevated serum cholesterol may be a risk factor for the formation of beta-amyloid plaques and for Alzheimer's disease. In addition, brain-specific factors that effect local brain cholesterol metabolism may also be implied in beta-amyloid formation (for a discussion e.g. Michikawa, (2003) J Neurosci Res 72(2):141-6; Tan et al (2003) Arch Intern Med 163(9):1053-7; Klsch al, (2003) J Nutr Health Aging 7(1):458-62); Buxbaum et al, (2001) J Alzheimers Dis (2):221-229).

Recent data also suggest that cholesterol in the brain may play a critical role in the generation of synapses among neurons (Mauch et al, (2001) Science 294(5545):1354-7). For example, large amounts of cholesterol turn over among glial cells and neurons during neuron repair and remodeling. If cholesterol indeed plays a fundamental role in the neuronal repair process, then the effects of disease or trauma to neurons (e.g., stroke, trauma, aging) may be modulated by compounds that regulate availability of cholesterol in brain.

Thus, a selective LXR modulator (SLRM) would indirectly act to promote local cholesterol availability from e.g. glial cells to proliferating cells like NSC and NPC- and an SLRM would also exert more direct actions on transcriptional control of proliferation and differentiation of NSC and the NPC. Thereby SLRMs promote formation of newborn neurons, i.e. neurogenesis, and in a novel fashion alleviate symptoms of a variety of CNS disorders where neurons are damaged, disorganized or dying.

### Production of Reagents

### Synthetic Low Molecular Weight Compounds

Reagents for treatment of patients can be produced, purified and formulated according to well known methods; e.g. by medicinal chemistry, parallel chemistry and combinatorial chemistry or any other synthetic organic chemistry application known in the art.

### Peptides

Reagents for treatment of patients can be recombinantly produced, purified and formulated according to well known methods.

Reagents of the invention and individual moieties or analogs and derivatives thereof, can be chemically synthesized. A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The peptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of peptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, e.g., receptor binding, functional potency or duration of action. See, e.g., Doherty, et al., 1993. J. Med. Chem. 36: 2585-2594; Kirby, et al., 1993, J. Med. Chem. 36:3802-3808; Morita, et al., 1994, FEBS Lett 353: 84-88; Wang, et al., 1993 Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the peptide backbone. This strategy can be used to develop peptide analogs of reagents with increased potency, selectivity and stability. A number of other methods have been used successfully to introduce conformational constraints into peptide sequences in order to improve their potency, receptor selectivity and biological half-life. These include the use of (i) C_{α}-methylamino acids (see, e.g., Rose, et al., Adv. Protein Chem. 37: 1-109 (1985); Prasad and Balaram, CRC Crit. Rev. Biochem., 16: 307-348 (1984)); (ii) N_{α}-methylamino acids (see, e.g., Aubry, et al., Int. J. Pept. Protein Res., 18: 195-202 (1981); Manavalan and Momany, Biopolymers, 19: 1943-1973 (1980)); and (iii) α,β-unsaturated amino acids (see, e.g., Bach and Gierasch, Biopolymers, 25: 5175-S192 (1986); Singh, et al., Biopolymers, 26: 819-829 (1987)). These and many other amino acid analogs are commercially available, or can be easily prepared. Additionally, replacement of the C-terminal acid with an amide can be used to enhance the solubility and clearance of a peptide.

Alternatively, a reagent may be obtained by methods well-known in the art for recombinant peptide expression and purification. A DNA molecule encoding the protein reagent can be generated. The DNA sequence is known or can be deduced from the protein sequence based on known codon usage. See, e.g., Old and Primrose, Principles of Gene Manipulation 3^{rd} ed., Blackwell Scientific Publications, 1985; Wada et al., Nucleic Acids Res. 20: 2111-2118(1992). Preferably, the DNA molecule includes additional sequence, e.g., recognition sites for restriction enzymes which facilitate its cloning into a suitable cloning vector, such as a plasmid. Nucleic acids may be DNA, RNA, or a combination thereof. Nucleic acids encoding the reagent may be obtained by any method known within the art (e.g., by PCR amplification using synthetic primers hybridizable to the 3′- and 5′-termini of the sequence and/or by cloning from a cDNA or genomic library using an oligonucleotide sequence specific for the given gene sequence, or the like). Nucleic acids can also be generated by chemical synthesis.

Any of the methodologies known within the relevant art regarding the insertion of nucleic acid fragments into a vector may be used to construct expression vectors that contain a chimeric gene comprised of the appropriate transcriptional/translational control signals and reagent-coding sequences. Promoter/enhancer sequences within expression vectors may use plant, animal, insect, or fungus regulatory sequences, as provided in the invention.

A host cell can be any prokaryotic or eukaryotic cell. For example, the peptide can be expressed in bacterial cells such as E. coli, insect cells, fungi or mammalian cells (such as Chinese hamster ovary cells (CHO) or COS cells). Other suitable host cells are known to those skilled in the art. In one embodiment, a nucleic acid encoding a reagent is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195).

The host cells, can be used to produce (e.g., overexpress) peptide in culture. Accordingly, the invention further provides methods for producing the peptide using the host cells of the invention. In one embodiment, the method comprises culturing the host cell of invention (into which a recombinant expression vector encoding the peptide has been introduced) in a suitable medium such that peptide is produced. The method further involves isolating peptide from the medium or the host cell. Ausubel et al., (Eds). In: Current Protocols in Molecular Biology. J. Wiley and Sons, New York, N.Y. 1998.

An “isolated” or “purified” recombinant peptide or biologically active portion thereof is substantially free of cellular material or other contaminating proteins from the cell or tissue source from which the peptide of interest is derived. The language “substantially free of cellular material” includes preparations in which the peptide is separated from cellular components of the cells from which it is isolated or recombinantly produced. In one embodiment, the language “substantially free of cellular material” includes preparations of peptide having less than about 30% (by dry weight) of peptide other than the desired peptide (also referred to herein as a “contaminating protein”), more preferably less than about 20% of contaminating protein, still more preferably less than about 10% of contaminating protein, and most preferably less than about 5% contaminating protein. When the peptide or biologically active portion thereof is recombinantly produced, it is also preferably substantially free of culture medium, e.g., culture medium represents less than about 20%, more preferably less than about 10%, and most preferably less than about 5% of the volume of the peptide preparation.

The invention also pertains to variants of a reagent that function as either agonists (mimetics) or as antagonists. Variants of a reagent can be generated by mutagenesis, e.g., discrete point mutations. An agonist of a reagent can retain substantially the same, or a subset of, the biological activities of the naturally occurring form of the reagent. An antagonist of the reagent can inhibit one or more of the activities of the naturally occurring form of the reagent by, for example, competitively binding to the receptor. Thus, specific biological effects can be elicited by treatment with a variant with a limited function. In one embodiment, treatment of a subject with a variant having a subset of the biological activities of the naturally occurring form of the reagent has fewer side effects in a subject relative to treatment with the naturally occurring form of the reagent.

Preferably, the analog, variant, or derivative reagent is functionally active. As utilized herein, the term “functionally active” refers to species displaying one or more known functional attributes of a full-length reagent. “Variant” refers to a reagent differing from naturally occurring reagent, but retaining essential properties thereof. Generally, variants are overall closely similar, and in many regions, identical to the naturally occurring reagent.

Variants of the reagent that function as either agonists (mimetics) or as antagonists can be identified by screening combinatorial libraries of mutants of the reagent for peptide agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential sequences is expressible as individual peptides, or alternatively, as a set of larger fusion proteins (e.g., for phage display) containing the set of sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl. Acids Res. 11:477.

Derivatives and analogs of the reagent or individual moieties can be produced by various methods known within the art. For example, the polypeptide sequences may be modified by any number of methods known within the art. See e.g., Sambrook, et al., 1990. Molecular Cloning: A Laboratory Manual, 2nd ed., (Cold Spring Harbor Laboratory Press; Cold Spring Harbor, N.Y.). Modifications include: glycosylation, acetylation, phosphorylation, amidation, derivatization by known protecting/blocking groups, linkage to an antibody molecule or other cellular reagent, and the like. Any of the numerous chemical modification methodologies known within the art may be utilized including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Derivatives and analogs may be full length or other than full length, if the derivative or analog contains a modified nucleic acid or amino acid, as described infra. Derivatives or analogs of the reagent include, but are not limited to, molecules comprising regions that are substantially homologous in various embodiments, of at least 30%, 40%, 50%, 60%, 70%, 80%, 90% or preferably 95% amino acid identity when: (i) compared to an amino acid sequence of identical size; (ii) compared to an aligned sequence in that the alignment is done by a computer homology program known within the art (e.g., Wisconsin GCG software) or (iii) the encoding nucleic acid is capable of hybridizing to a sequence encoding the aforementioned peptides under stringent (preferred), moderately stringent, or non-stringent conditions. See, e.g., Ausubel, et al., Current Protocols in Molecular Biology, John Wiley and Sons, New York, N.Y., 1993.

Derivatives of the reagent may be produced by alteration of their sequences by substitutions, additions or deletions that result in functionally-equivalent molecules. One or more amino acid residues within the reagent may be substituted by another amino acid of a similar polarity and net charge, thus resulting in a silent alteration. Conservative substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid.

The reagent can be administered locally to any loci implicated in the CNS disorder pathology, e.g. any loci deficient in neural cells as a cause of the disease. For example, the reagent can be administered locally to the ventricle of the brain, substantia nigra, striatum, locus ceruleous, nucleus basalis of Meynert, pedunculopontine nucleus, cerebral cortex, spinal cord and retina.

Neural stem cells and their progeny can be induced to proliferate, differentiate, survive or migrate in vivo by administering to the host a reagent, alone or in combination with other agents, or by administering a pharmaceutical composition containing the reagent that will induce proliferation and differentiation of the cells. Pharmaceutical compositions include any substance that blocks the inhibitory influence and/or stimulates neural stem cells and stem cell progeny to proliferate, differentiate, migrate and/or survive. Such in vivo manipulation and modification of these cells allows cells lost, due to injury or disease, to be endogenously replaced, thus obviating the need for transplanting foreign cells into a patient.

### Antibodies

Included in the invention are antibodies to be used as reagents. The term “antibody” as used herein refers to immunoglobulin molecules and immunologically active portions of immunoglobulin (Ig) molecules, e.g., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an Fab expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic site is a region of an LXR-activator, an SLRM, a carrier of an LXR-activator or an LXR that is located on the surface of the protein, e.g., a hydrophilic region. A hydrophobicity analysis of the human variant of those protein sequences will indicate which regions of the polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein or other determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An LXR-activator, an SLRM, a carrier of an LXR-activator or an LXR polypeptide or a fragment thereof comprises at least one antigenic epitope. An anti-LXR-activator, -SLRM, -carrier of an LXR-activator or -LXR antibody of the present invention is said to specifically bind to the antigen when the equilibrium binding constant (KD) is ≦1 μM, preferably ≦100 nM, more preferably ≦10 nM, and most preferably ≦100 pM to about 1 pM, as measured by assays such as radioligand binding assays or similar assays known to those skilled in the art.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

### Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp.25-28).

### Monoclonal Antibodies

The term “monoclonal antibody” (MAb) or “monoclonal antibody composition,” as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

### Humanized Antibodies

The antibodies directed against the protein and other antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

### Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

### Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA or TETA. Another bispecific antibody of interest binds the protein antigen described herein and further binds tissue factor (TF).

### Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction.

### Antibody Therapeutics

Antibodies of the invention, including polyclonal, monoclonal, humanized and fully human antibodies, may be used as therapeutic agents such as one of this invention. Such agents will generally be employed to treat or prevent a disease or pathology, specifically neurological disease, in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous LXR ligand to which it naturally binds. In this case, the antibody binds to the target and masks a binding site of the naturally occurring ligand, wherein the ligand serves as an effector molecule. Thus, the receptor mediates a signal transduction pathway for which ligand is responsible.

Alternatively, the effect may be one in which the antibody elicits a physiological result by virtue of binding to an effector binding site on the target molecule. In this case the target, LXRr having an endogenous ligand which needs to be modulated, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen and the rate at which an administered antibody is depleted from the free volume of the subject to which it is administered.

### Diseases and Disorders

Diseases and disorders that are characterized by altered (relative to a subject not suffering from the disease or disorder) levels or biological activity may be treated with therapeutics that antagonize (e.g., reduce or inhibit) or activate LXR or RXR activity. Therapeutics that antagonize activity may be administered in a therapeutic or prophylactic manner. Therapeutics that may be utilized include, but are not limited to: (i) an LXR activator, SLRM, analog, derivatives, fragments or homologs thereof; (ii) antibodies to an aforementioned reagent; (iii) administration of antisense nucleic acid and nucleic acids that are “dysfunctional” (e.g., due to a heterologous insertion within the coding sequences of coding sequences to an aforementioned peptide) that are utilized to “knockout” endogenous function of an aforementioned LXR by homologous recombination (see, e.g., Capecchi, 1989. Science 244: 1288-1292); or (iv) modulators (e.g., inhibitors, agonists and antagonists, including additional mimetics of the invention or antibodies specific to a peptide or reagent or carrier of an endogenous activator of the invention) that alter the interaction between an aforementioned reagent and its binding partner.

Diseases and disorders that are characterized by altered (relative to a subject not suffering from the disease or disorder) levels or biological activity may be treated with therapeutics that increase (e.g., are agonists to) activity. In a preferred embodiment, the diseases to be treated include e.g. Alzheimer's disease, stroke, Parkinson's disease. Therapeutics that upregulate activity may be administered in a therapeutic or prophylactic manner. Therapeutics that may be utilized include, but are not limited to, an aforementioned reagent, analog, derivatives, fragments or homologs thereof; or an agonist that increases bioavailability.

Increased or decreased levels can be detected by quantifying reagent, peptide and/or RNA, by obtaining a patient tissue sample (e.g., from biopsy tissue) and assaying it in vitro for RNA or peptide or reagent levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like) and/or mass-spectrometry linked to chromatography.

### Therapeutic Methods

Another aspect of the invention pertains to methods of modulating LXR activator or modulator expression or activity for therapeutic purposes. In one embodiment, the modulatory method of the invention involves contacting a cell with an agent that modulates one or more of the activities of an LXR activator or modulator. An agent that modulates this activity can be an agent as described herein, such as a nucleic acid or a protein, a naturally-occurring cognate ligand of a an LXR ligand, a peptide, a mimetic, or other small molecule. In one embodiment, the agent stimulates the activity of the LXR signaling pathway. Examples of such stimulatory agents include active LXR ligand and a nucleic acid molecule encoding the LXRs that has been introduced into the cell. These modulatory methods can be performed in vitro (e.g., by culturing the cell with the agent) or, alternatively, in vivo (e.g., by administering the agent to a subject). As such, the invention provides methods of treating an individual afflicted with a disease or disorder, specifically a neurological disorder. In one embodiment, the method involves administering a reagent (e.g., an reagent identified by a screening assay described herein), or combination of reagents that modulate (e.g., up-regulates or down-regulates) LXR ligand expression or activity. In another embodiment, the method involves administering LXR ligands as therapy to modulate proliferation, differentiation, migration and/or survival of NSC.

Stimulation of LXR ligand activity is desirable in situations in which endogenous LXR ligand or activator are abnormally downregulated and/or in which increased LXR activator activity is likely to have a beneficial effect. One example of such a situation is where a subject has a disorder characterized by aberrant cell proliferation and/or differentiation (e.g., Parkinson's disease and Alzheimer's disease).

### Determination of the Biological Effect of the Therapeutic

In various embodiments of the invention, suitable in vitro or in vivo assays are performed to determine the effect of a specific therapeutic and whether its administration is indicated for treatment of the affected tissue.

In various specific embodiments, in vitro assays may be performed with representative stem cells or newly differentiated cells involved in the patient's disorder, to determine if a given therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Pharmaceutical Compositions

The invention provides methods of influencing central nervous system cells to produce progeny that can replace damaged or missing neurons in the central nervous system or other central nervous system cell types by exposing a patient, suffering from a neurological disease or disorder, to a reagent (e.g. an LXR ligand, -activator or SLRM) in a suitable formulation through a suitable route of administration, that modulates NSC or NPC activity in vivo. In all embodiment of the inventions, the reference to disease or disorder of the nervous system may include any disorder and, for example, at least the following disorders: neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders. In one embodiment of the invention, the disease or disorder of the nervous system is selected from the group consisting of Parkinson's disease and Parkinsonian disorders, Huntington's disease, Alzheimer's disease, Amyotrophic Lateral Sclerosis, spinal ischemia, ischemic stroke, spinal cord injury and cancer-related brain/spinal cord injury. In a further embodiment of the invention, the disease or disorder of the nervous system is selected from the group consisting of schizophrenia and other psychoses, lissencephaly syndrome, depression, bipolar depression/disorder, anxiety syndromes/disorders, phobias, stress and related syndromes, cognitive function disorders, aggression, drug and alcohol abuse, obsessive compulsive behavior syndromes, seasonal mood disorder, borderline personality disorder, cerebral palsy, life style drug, multi-infarct dementia, Lewy body dementia, age related/geriatric dementia, epilepsy and injury related to epilepsy, temporal lobe epilepsy, spinal cord injury, brain injury, brain surgery, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy, sleep disorders, and disorders of cognitive performance or memory.

This invention provides a method of treating a neurological disease or disorder comprising administering a reagent that modulates neural stem cell or neural progenitor cell activity in vivo to a mammal. The term “mammal” refers to any mammal classified as a mammal, including humans, cows, horses, dogs, sheep and cats. In one embodiment, the mammal is a human.

The invention provides a regenerative cure for neurodegenerative diseases by stimulating ependymal cells and subventricular zone cells to proliferate, migrate, differentiate and survive into the desired neural phenotype targeting loci where cells are damaged or missing. In vivo stimulation of ependymal stem cells is accomplished by locally administering a reagent to the cells in an appropriate formulation. By increasing neurogenesis, damaged or missing neurons can be replaced in order to enhance brain function in diseased states.

A pharmaceutical composition useful as a therapeutic agent for the treatment of central nervous system disorders is provided. For example, the composition includes a reagent of the invention, which can be administered alone or in combination with the systemic or local co-administration of one or more additional agents. Such agents include preservatives, ventricle wall permeability increasing factors, stem cell mitogens, survival factors, glial lineage preventing agents, anti-apoptotic agents, anti-stress medications, neuroprotectants, and anti-pyrogenics. The pharmaceutical composition preferentially treats CNS diseases by stimulating cells (e.g., ependymal cells and subventricular zone cells) to proliferate, migrate and differentiate into the desired neural phenotype, targeting loci where cells are damaged or missing.

A method for treating a subject suffering from a CNS disease or disorder is also provided. This method comprises administering to the subject an effective amount of a pharmaceutical composition containing a reagent (1) alone in a dosage range of 0.001 ng/kg/day to 100 mg/kg/day, preferably in a dosage range of 0.1 ng/kg/day to 100 mg/kg/day, more preferably in a dosage range of 1 ng/kg/day to 50 mg/kg/day, most preferably in a dosage range of 50 ng/kg/day to 50 mg/kg/day, (2) in a combination with a ventricle wall permeability increasing factor, or (3) in combination with a locally or systemically co-administered agent.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that it can be handled with a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., chimeric peptide) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The reagents or compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Nucleic acid molecules encoding a proteinaceous agent can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

In another embodiments, the reagent is administered in a composition comprising at least 90% pure reagent. The reagent can be, for example an LXR activator or an SLRM.

Preferably the reagent is formulated in a medium providing maximum stability and the least formulation-related side-effects. In addition to the reagent, the composition of the invention will typically include one or more protein carrier, buffer, isotonic salt and stabilizer.

In some instances, the reagent can be administered by a surgical procedure implanting a catheter coupled to a pump device. The pump device can also be implanted or be extracorporally positioned. Administration of the reagent can be in intermittent pulses or as a continuous infusion. Devices for injection to discrete areas of the brain are known in the art (see, e.g., U.S. Pat. Nos. 6,042,579; 5,832,932; and 4,692,147).

Reagents containing compositions can be administered in any conventional form for administration of a protein. A reagent can be administered in any manner known in the art in which it may either pass through or by-pass the blood-brain barrier. Methods for allowing factors to pass through the blood-brain barrier include minimizing the size of the factor, providing hydrophobic factors which may pass through more easily, conjugating the protein reagent or other agent to a carrier molecule that has a substantial permeability coefficient across the blood brain barrier (see, e.g., U.S. Pat. No. 5,670,477).

Reagents, derivatives, and co-administered agents can be incorporated into pharmaceutical compositions suitable for administration. Such compositions typically comprise the agent and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the agents to affect solubility or clearance of the peptide. Peptidic molecules may also be synthesized with D-amino acids to increase resistance to enzymatic degradation. In some cases, the composition can be co-administered with one or more solubilizing agents, preservatives, and permeation enhancing agents. Examples of pharmaceutically acceptable carriers include lactose, glucose, sucrose, sorbitol, mannitol, corn starch, crystalline cellulose, gum arabic, calcium phosphate, alginates, calcium silicate, microcrystalline cellulose, polyvinyl pyrrolidone, tragacanth gum, gelatin, syrup, methyl cellulose, carboxymethyl cellulose, methylhydroxybenzoic acid esters, propylhydroxybenzoic acid esters, talc, magnesium stearates, inert polymers, water and mineral oils.

For example, the composition can include a preservative or a carrier such as proteins, carbohydrates, and compounds to increase the density of the pharmaceutical composition. The composition can also include isotonic salts and redox-control agents.

In some embodiments, the composition administered includes the reagent and one or more agents that increase the permeability of the ventricle wall, e.g. “ventricle wall permeability enhancers.” Such a composition can help an injected composition penetrate deeper than the ventricle wall. Examples of suitable ventricle wall permeability enhancers include, for example, liposomes, VEGF (vascular endothelial growth factor), IL-s, TNFα, polyoxyethylene, polyoxyethylene ethers of fatty acids, sorbitan monooleate, sorbitan monolaurate, polyoxyethylene monolaurate, polyoxyethylene sorbitan monolaurate, fusidic acid and derivatives thereof, EDTA, disodium EDTA, cholic acid and derivatives, deoxycholic acid, glycocholic acid, glycodeoxycholic acid, taurocholic acid, taurodeoxycholic acid, sodium cholate, sodium glycocholate, glycocholate, sodium deoxycholate, sodium taurocholate, sodium glycodeoxycholate, sodium taurodeoxycholate, chenodeoxycholic acid, urosdeoxycholic acid, saponins, glycyrrhizic acid, ammonium glycyrrhizide, decamethonium, decamethonium bromide, dodecyltrimethylammonium bromide, and dimethyl-β-cyclodextrin or other cyclodextrins.

### Drug Screening

The invention also provides a method of using the receptors or receptor/reagent complexes for analyzing or purifying certain stem or progenitor cell populations, using e.g. antibodies, against the receptors or receptor/reagent complexes.

In another aspect, the invention provides a method for screening for reagents that influence stem and progenitor cells. In some applications, neural cells (undifferentiated or differentiated) are used to screen factors that promote maturation into neural cells, or promote proliferation and maintenance of such cells in long-term culture. For example, candidate reagents are tested by adding them to cells in culture at varying dosages, and then determining any changes that result, according to desirable criteria for further culture and use of the cells. Physical characteristics of the cells can be analyzed by observing cell and neurite growth with microscopy. The induction of expression of increased levels of proliferation, differentiation and migration can be analyzed with any technique known in the art which can identify proliferation and differentiation. Such techniques include RT-PCR, in situ hybridization, and ELISA.

In one aspect, novel receptor/reagents in undifferentiated neurospheres was examined using RT-PCR techniques. In particular, genes that are up-regulated in these undifferentiated neurospheres were identified. As used herein, the term “up-regulation” refers to a process that increases reagent/receptor interactions due to an increase in the number of available receptors. The presence of these genes suggests a potential role in the mediation of signal transduction pathways in the regulation of NSC function. Furthermore, by knowing the levels of expression of the receptors or their various reagents, it is possible to diagnose disease or determine the role of stem and progenitor cells in the disease. By analyzing the genetic or amino-acid sequence variations in these genes or gene products, it is possible to diagnose or predict the development of certain diseases. Such analysis will provide the necessary information to determine the usefulness of using stem or progenitor cell based treatments for disease.

In another aspect, in situ hybridization is performed on adult mouse brain sections to determine which cells in the adult brain express these signaling pathways. This data is helpful in determining treatment options for various neurological diseases.

To determine the effect of a potential reagent on neural cells, a culture of NSC derived from multipotent stem cells can be obtained from normal neural tissue or, alternatively, from a host afflicted with a CNS disease or disorder. The choice of culture will depend upon the particular agent being tested and the effects one wishes to achieve. Once the cells are obtained from the desired donor tissue, they are proliferated in vitro in the presence of a proliferation-inducing reagent.

The ability of various biological agents to increase, decrease or modify in some other way the number and nature of the stem cell progeny proliferated in the presence of the proliferative factor can be screened on cells proliferated by the methods previously discussed. For example, it is possible to screen for reagents that increase or decrease the proliferative ability of NSC which would be useful for generating large numbers of cells for transplantable purposes. In these studies precursor cells are plated in the presence of the reagent in question and assayed for the degree of proliferation and survival or progenitor cells and their progeny can be determined. It is possible to screen neural cells which have already been induced to differentiate prior to the screening. It is also possible to determine the effects of the reagent on the differentiation process by applying them to precursors cells prior to differentiation. Generally, the reagent will be solubilized and added to the culture medium at varying concentrations to determine the effect of the agent at each dose. The culture medium may be replenished with the reagent every couple of days in amounts so as to keep the concentration of the reagent somewhat constant.

Changes in proliferation are observed by an increase or decrease in the number of neurospheres that form and/or an increase or decrease in the size of the neurospheres, which is a reflection of the rate of proliferation and is determined by the numbers of precursor cells per neurosphere.

Using these screening methods, it is possible to screen for potential drug side-effects on prenatal and postnatal CNS cells by testing for the effects of the biological agents on stem cell and progenitor cell proliferation and on progenitor cell differentiation or the survival and function of differentiated CNS cells.

Other screening applications of this invention relate to the testing of pharmaceutical compounds for their effect on neural tissue. Screening may be done either because the compound is designed to have a pharmacological effect on neural cells, or because a compound designed to have effects elsewhere may have unintended side effects on the nervous system. The screening can be conducted using any of the neural precursor cells or terminally differentiated cells of the invention.

Effect of cell function can be assessed using any standard assay to observe phenotype or activity of neural cells, such as receptor binding, proliferation, differentiation, survival-either in cell culture or in an appropriate model.

### Therapeutic Uses, Local Administration

The fact that neural stem cells are located in the tissues lining ventricles of mature brains offers several advantages for the modification and manipulation of these cells in vivo and the ultimate treatment of various neurological diseases, disorders, and injury that affect different regions of the CNS. Therapy for these diseases can be tailored accordingly so that stem cells surrounding ventricles near the affected region would be manipulated or modified in vivo using the methods described herein. The ventricular system is found in nearly all brain regions and thus allows easier access to the affected areas. In order to modify the stem cells in vivo by exposing them to a composition comprising a reagent, it is relatively easy to implant a device that administers the composition to the ventricle and thus, to the neural stem cells. For example, a cannula attached to an osmotic pump may be used to deliver the composition. Alternatively, the composition may be injected directly into the ventricles. The neural stem cell progeny can migrate into regions that have been damaged as a result of injury or disease. Furthermore, the close proximity of the ventricles to many brain regions would allow for the diffusion of a secreted neurological agent by the stem cells or their progeny.

In an additional embodiment, a reagent of the invention is administered locally, as described above, in combination with an agent administered locally or systemically. Such agents include, for example, one or more stem cell mitogens, survival factors, glial-lineage preventing agents, anti-apoptotic agents, anti-stress medications, neuroprotectants, and anti-pyrogenics, or any combination thereof.

The agent is administered systemically before, during, or after administration of the reagent of the invention. The locally administered agent can be administered before, during, or after the reagent administration.

For treatment of Huntington's Disease, Alzheimer's Disease, Parkinson's Disease, and other neurological disorders affecting primarily the forebrain, a reagent alone or with an additional agent or agents is delivered to the ventricles of the forebrain to affect in vivo modification or manipulation of the stem cells. For example, Parkinson's Disease is the result of low levels of dopamine in the brain, particularly the striatum. It is therefore advantageous to induce a patient's own quiescent stem cells to begin to divide in vivo and to induce the progeny of these cells to differentiate into dopaminergic cells in the affected region of the striatum, thus locally raising the levels of dopamine.

Normally the cell bodies of dopaminergic neurons are located in the substantia nigra and adjacent regions of the mesencephalon, with the axons projecting to the striatum. The methods and compositions of the invention provide an alternative to the use of drugs and the controversial use of large quantities of embryonic tissue for treatment of Parkinson's disease. Dopamine cells can be generated in the striatum by the administration of a composition comprising a reagent of the invention to the lateral ventricle.

For the treatment of MS and other demyelinating or hypomyelinating disorders, and for the treatment of Amyotrophic Lateral Sclerosis or other motor neuron diseases, a reagent of the invention, alone or with an additional agent or agents is delivered to the central canal.

In addition to treating CNS tissue immediately surrounding a ventricle, a reagent of the invention, alone or with an additional agent or agents can be administered to the lumbar cistern for circulation throughout the CNS.

In other aspects, neuroprotectants can also be co-administered systemically or locally before, during and/or after infusion of a regent of the invention. Neuroprotectants include antioxidants (agents with reducing activity, e.g., selenium, vitamin E, vitamin C, glutathione, cysteine, flavinoids, quinolines, enzymes with reducing activity, etc), Ca-channel modulators, Na-channel modulators, glutamate receptor modulators, serotonin receptor agonists, phospholipids, unsaturated- and polyunsaturated fatty acids, estrogens and selective estrogen receptor modulators (SERMS), progestins, thyroid hormone and thyroid hormone-mimicking compounds, cyclosporin A and derivatives, thalidomide and derivatives, methylxanthines, MAO inhibitors; serotonin-, noradrenaline and dopamine uptake blockers; dopamine agonists, L-DOPA, nicotine and derivatives, and NO synthase modulators.

In another aspect of the invention, anti-apoptotic agents including caspase inhibitors and agents useful for antisense-modulation of apoptotic enzymes and factors can be administered before, during, or after administration of the reagent of the invention.

Stress syndromes lower neurogenesis, therefore in some aspects, it may be desirable to treat a subject with anti-stress medications such as, e.g., anti-glucocorticoids (e.g., RU486) and beta-blockers, administered systemically or locally before, during and/or after administration of the reagent of the invention.

Methods for preparing the reagent dosage forms are known, or will be apparent, to those skilled in this art.

The amount of reagent to be administered will depend upon the exact size and condition of the patient, but will be from 0.5 ng/kg/day to 5000 ng/kg/day in a volume of 0.001 to 10 ml.

The duration of treatment and time period of administration of reagent will also vary according to the size and condition of the patient, the severity of the illness and the specific composition and method being used.

The effectiveness of each of the foregoing methods for treating a patient with a CNS disease or disorder is assessed by any known standardized test for evaluating the disease.

### Specific Embodiments

One embodiment of the invention is directed to a method of alleviating a symptom of a disorder of the nervous system in a patient by administering a “NSC therapeutic agent” to the patient. The NSC therapeutic agent is may be an LXR activator or a SLRM. Administration of the NSC therapeutic agent modulates a NSC activity (proliferation, differentiation, migration, or survival) in vivo to alleviate the symptom.

The NSC therapeutic agent may be administered in a dose between 0.001 ng/kg/day to 100 mg/kg/day, other suitable dose ranges are 0.1 ng/kg/day to 100 mg/kg/day, 1 ng/kg/day to 50 mg/kg/day and 50 ng/kg/day to 50 mg/kg/day.

Another method for determining proper dosage is to administering sufficient NSC therapeutic agents to achieve a target tissue concentration of 0.01 nM to 50 μM. The target tissue to be monitor could be any neural or CNS tissue, including, at least, the ventricular wall, the volume adjacent to the wall of the ventricular system, hippocampus, alveus, striatum, substantia nigra, retina, nucleus basalis of Meynert, spinal cord, thalamus, hypothalamus and cortex. Other suitable target tissue includes a region of tissue that is impaired by stroke injury or ischemic injury.

Administration of any of the NSC therapeutic agents, may be performed by injection. The method of injection include subcutaneous, intraperitoneal, intramusclular, intracerebroventricular, intraparenchymal, intrathecal or intracranial injection. In another embodiment, the NSC therapeutic agents may be administered orally. Other suitable administrations means include administration to the buccal, nasal or rectal mucosa. In addition, NSC therapeutic agents may be administered by via peptide fusion or micelle delivery.

The disorders that can be treated by the methods of the invention includes any disorder listed in this disclosure. These disorders may be classified to include, at least, neurodegenerative disorders, NSC disorders, neural progenitor disorders, ischemic disorders, neurological traumas, affective disorders, neuropsychiatric disorders, degenerative diseases of the retina, retinal injury/trauma, cognitive performance and learning and memory disorders.

Another embodiment of the invention is directed to a method of modulating the activity of a receptor SLRMs or the LXRs. The method involves exposing the cell expressing the receptor to a modulator agent so that the exposure induces NSC to proliferate, differentiate, migrate or survive. In this embodiment, the modulator agent may be an exogenous reagent, an antibody (monoclonal, polyclonal, or an engineered antibody), an affibody or a combination of these agents. The LXRs, which are targeted for contact by the modulator agent may be LXRalpha (or NR1H3) and LXRbeta (or NR1H2).

The modulator agent may be reagents as described above that are LXR activators or SLRMs. Furthermore, the modulator agent may be pegylated to enhance its stability and efficacy in patients. Methods of pegylating reagents are well known to those of skill in the art and are described, for example, in U.S. Pat. Nos. 5,166,322, 5,766,897, 6,420,339 and 6,552,170.

In a preferred embodiment, the NSC is derived from fetal brain, adult brain, neural cell culture or a neurosphere. In another preferred embodiment, the NSC is derived from tissue enclosed by dura mater, peripheral nerves or ganglia. Other examples of suitable NSC include NSC derived from stem cells originating from pancreas, skin, muscle, adult bone marrow, liver, umbilical cord tissue or umbilical cord blood.

Another embodiment of the invention is directed to a method of stimulating mammalian adult NSC to proliferate, to undergo neurogenesis, to differentiate or to migrate. In the method, the adult NSC cells are contacted to LRX activator or SLRM to form a treated NSC cell that has improved proliferation, neurogenesis, migration, or differentiation properties compared to untreated cells. The NSC cells may be derived from lateral ventricle wall of a mammalian brain. In a preferred embodiment, the NSC is derived from stem cells from pancreas, skin, muscle, adult bone marrow, liver, umbilical cord tissue or umbilical cord blood.

Another embodiment of the invention is directed to a method for synergistically stimulation of mammalian adult NSC proliferation and neurogenesis. In the method, a mammalian adult NSC is contacted to a growth factor and a NSC therapeutic agent. The two reagents induces the mammalian induce the NSC cell to proliferate at a rate greater than either the growth factor or NSC therapeutic agent alone. In a surprising discovery, the combination of growth factor and NSC therapeutic agent has a synergistic effect that is greater that the sum of growth factor effect and NSC therapeutic agent effect. In a preferred embodiment, the growth factor for use in this method is EGF.

Another embodiment of the invention is directed to a method for cooperative stimulation of mammalian adult NSC proliferation and neurogenesis. In the method, a mammalian adult NSC is contacted to a growth factor and a NSC therapeutic agent. The two reagents induces the mammalian induce the NSC cell to proliferate at a rate greater than either the growth factor or NSC therapeutic agent alone. In a preferred embodiment, the growth factor for use in this method is VEGF and PDGF.

Another embodiment of the invention is directed to a method of stimulating mammalian adult NSC proliferation. The method involves contacting a NSC therapeutic agent to a mammalian adult NSC. The NSC therapeutic agent induces a modulation of intracellular CREB phosphorylation. Mammalian adult NSC proliferation is, in turn, induced by the intracellular CREB phosphorylation.

Another embodiment of the invention is directed to a method of stimulating mammalian adult NSC proliferation. The method involves contacting a NSC therapeutic agent to a mammalian adult NSC. The NSC therapeutic agent induces a modulation of intracellular AP-1 transcription. Mammalian adult NSC proliferation is, in turn, induced by the intracellular AP-1 transcription.

Another embodiment of the invention is directed to a method of stimulating mammalian adult NSC proliferation. The method involves contacting a NSC therapeutic agent to a mammalian adult NSC. The NSC therapeutic agent induces a modulation of intracellular protein kinase C activity. Mammalian adult NSC proliferation is, in turn, induced by the intracellular protein kinase C activity.

Another embodiment of the invention is directed to a method for stimulating survival of mammalian adult NSC progeny by contacting the NSC cell with a NSC therapeutic agent. The increase in survival of NSC progeny may be characterized and predicted by (1) modulation of intracellular CREB phosphorylation, (2) modulation of intracellular AP-1 transcription, (3) modulation of intracellular protein kinase C activity and (4) modulation of intracellular protein kinase A activity. Mammalian adult NSC progeny survival can be mediated by stimulating any of the four characteristics listed above by the use of a NSC therapeutic agent.

Another embodiment of the invention is directed to a method of stimulating primary adult mammalian NSC to proliferate to form neurospheres. In the method, adult mammalian NSC is contacted with a NSC therapeutic agent to cause the cells to proliferate and form neurospheres.

Another embodiment of the invention is directed to a method for reducing a symptom of a central nervous system disorder in a mammal by administering a NSC therapeutic agent to the mammal. The agonist or activator may be, for example, an antibody, an affibody, a small molecule, peptide and a receptor. The receptor may be an LXR. In a preferred embodiment, the administration may be local or systemic. Further, the administration may include a ventricle wall permeability enhancer. The ventrical wall permeability enhancer may be administered before or after the NSC therapeutic agent. In a preferred embodiment, the NSC therapeutic agent is mixed with the permeability enhancer and a pharmaceutically acceptable carrier and administered. In a preferred embodiment, the method is enhanced by a further administration of stem cell mitogens, survival factors, glial-lineage preventing agents, anti-apoptotic agents, anti-stress medications, neuroprotectants, anti-pyrogenics, differentiation factors and a combination thereof.

Another embodiment of the invention is directed to a method for inducing the in situ proliferation, differentiation, migration or survival of a NSC located in the neural tissue of a mammal. The method involves administering a therapeutically effective amount of a NSC therapeutic agent to the neural tissue to induce the proliferation, differentiation, migration or survival of the NSC.

Another embodiment of the invention is directed to a method for accelerating the growth of NSC in a desired target tissue in a patient. In the method, a target tissue is transfected with an expression vector containing an open reading frame encoding LXRalpha (or NR1H3) and LXRbeta (or NR1H2) gene in a therapeutically effective amount. The expression vector directs the expression of the open reading frame and the expressed protein accelerate the growth of the NSC in the target tissue. One advantage of this method is that while all, or most, of the cells in the targeted tissue is transfected, only the NSC cells are induced to accelerate the growth. The transfection step may be performed by administration of the expression vector by injection. Any of the injection methods described in this disclosure may be used. These method includes, at least, subcutaneous, intraperitoneal, intramuscular, intracerebroventricular, intraparenchymal, intrathecal, intracranial injection. The expression vector may be, for example, a non-viral expression vector encapsulated in a liposome.

Another embodiment of the invention is directed to a method of enhancing neurogenesis in a patient suffering from a central nervous system disorder by infusing a NSC therapeutic agent into the patient.

Another embodiment of the invention is directed to a method of alleviating a symptom of a central nervous system disorder in a patient by infusing LXR activator or SLRM into the patient.

Another embodiment of the invention is directed to a method for producing a cell population enriched for human NSC. The method involves contacting a cell population with NSC with a reagent that specifically bind a determinant on a receptor for an LXR activator or SLRM. Then cells in which there is contact between the reagent and the determinant of the cells of the previous step is selected to produce a population highly enriched for central nervous system stem cells. The reagent may be a small molecule, a peptide, an antibody and an affibody. In one embodiment the population containing NSC are obtained from a neural tissue progenitor cell. A neural tissue progenitor cell is any population of cells which gives rise to neural tissue. For example, the cell population may be a cell population derived from whole mammalian fetal brain or whole mammalian adult brain. Further, the human NSC may be derived from stem cells originating from a tissue such as pancreas, skin, muscle, adult bone marrow, liver, umbilical cord tissue and umbilical cord blood. The described method is useful for enriching for cells expressing receptors such as LXRalpha (or NR1H3) and LXRbeta (or NR1H2).

Another embodiment of the invention is directed to an in vitro cell culture comprising a cell population generated by the method of the previous paragraph. Another embodiment of the invention is directed to a method for alleviating a symptom of a central nervous system disorder comprising administering the cells to a mammal exhibiting the symptom. A non-human mammal engrafted with the human NSC made by the method is also envisioned. The non-human mammal may be, for example, a rat, mouse, rabbit, horse, sheep, pig or guinea pig.

Another embodiment of the invention is directed to a method for reducing a symptom of a CNS disorder in a patient with the step of administering into the spinal cord of the patient a composition with a population of isolated NSC obtained from fetal or adult tissue; and a NSC therapeutic agent.

Another embodiment of the invention is directed to a method of reducing a symptom of a central nervous disorder in a patient. In the method, a viral vector for expressing a NSC therapeutic agent is introduced into a target cell. For expression, the viral vector may have at least one insertion site containing a nucleic acid which encoded a NSC therapeutic agent linked to a promoter capable of expression in the host cell (i.e., target cell). The NSC therapeutic agent is expressed to produce a protein in a target cell to reduce said symptom. In a preferred embodiment, the viral vector is a non-lytic viral vector.

Another embodiment of the invention is directed to a method of gene delivery and expression in a target cell of a mammal. The method comprise providing a nucleic acid molecule encoding a NSC therapeutic agent, selecting a viral vector with for insertion of the isolated nucleic acid molecule so that the molecule can be operably linked to a promoter capable of expression in the target cells, inserting the isolated nucleic acid fragment into the insertion site, and introducing the vector into the target cell wherein the gene is expressed at detectable levels. The virus may be, for example, a retrovirus, adenovirus, pox virus (vaccinia), iridoviruses, coronaviruses, togaviruses, caliciviruses, lentiviruses, adeno-associated viruses or picornaviruses. In a preferred embodiment, the virus strain is genetically modified to be non-virulent in a host.

Another embodiment of the invention is directed to a method for alleviating a symptom of a disorder of the nervous system with the steps of providing a population of NSC, suspending the NSC in a solution comprising LXR activator or SLRM to create a cell suspension, and delivering the cell suspension to an injection site in the nervous system of the patient to alleviate the symptom. In addition, a further step of administering to the injection site a growth factor for a period of time before or after the step of delivering the cell suspension may be added.

Another embodiment of the invention is directed to a method for transplanting a population of cells enriched for human NSC, comprising the steps of contact a population containing NSC with a reagent that recognizes a determinant on an LXR, selecting for cells in which there is contacted between the reagent and the determinant of the cells of the previous step to produce a population highly enriched for central nervous system stem cells; and implanting the selected cells into a non-human mammal.

Another embodiment of the invention is directed to a method of modulating a receptor for an LXR activator or SLRM in an NSC using the step of contacting the cell expressing the receptor to exogenous reagent, antibody, or affibody so that the exposure induces the NSC to proliferation, differentiation, migration or survival. In this embodiment the NSC may be derived from fetal brain, adult brain, neural cell culture or a neurosphere.

Another embodiment of the invention is directed to a method for testing an isolated candidate SLRM for its ability to modulate NSC activity. In the method, the isolated compound is administered to a non-human mammal; and it is determined if the candidate compound has an effect on modulating the NSC activity in the non-human mammal. The determining step may involve comparing the neurological effects of said non-human mammal with a referenced non-human mammal not administered the candidate compound. The NSC activity may be proliferation, differentiation, migration or survival. Administration may be performed, for example by injection using any method including peptide fusion or micelle delivery, discussed in this disclosure.

Other features of the invention will become apparent in the course of the following description of exemplary embodiments which are given for illustration of the invention and are not intended to be limiting thereof. All references, patents and patent applications cited are hereby incorporated by reference in their entirety.

### EXAMPLES

### Example 1

### Expression of LXRα and LXRβ Genes in Adult Mouse Neurosphere Cultures

Methods

A. Mouse Neurosphere Cultures.

The anterior lateral wall of the lateral ventricle of 5-6 week old mice was enzymatically dissociated in 0.8 mg/ml hyaluronidase and 0.5 mg/ml trypsin in DMEM containing 4.5 mg/ml glucose and 80 units/ml DNase at 37° C. for 20 min. The cells were gently triturated and mixed with three volumes of Neurosphere medium (DMEM/F12, B27 supplement, 12.5 mM HEPES pH7.4) containing 20 ng/ml EGF (unless otherwise stated), 100 units/ml penicillin and 100 μg/ml streptomycin. After passing through a 70 μm strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split neurosphere cultures, neurospheres were collected by centrifugation at 160×g for 5 min. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, resuspended in fresh Neurosphere medium supplemented with 20 ng/ml EGF and 1 nM bFGF plated out and incubated at 37° C.

B. Library Construction.

Total RNA, mRNA and cDNA were generated from the anterior lateral wall of the lateral ventricle of 5-6 week old mice or from mouse neruospheres, using Qiagen shredder (Qiagen), RNAeasy (Qiagen), Oligo-dT and poly-T primed cDNA (InVitroGen 2000, Invitrogen), all according to the manufacturer's instructions. The resulting cDNAs were subcloned by blunt-end ligation into pCMVSPORT6 (creating SalI and NotI adaptors) to create the anterior lateral ventricle wall library. The library was sequenced.

C. RT-PCR

Mouse Neurospheres:

The following primer pairs were designed to specifically identify the presence of LXRα and LXRβ gene expression in mouse neurospheres and lateral ventricle wall tissue. Estimated band sizes for each primer pair are given below:

Band
size
(base
pairs)

| | | | |
|---|---|---|---|
| LXRα | GTGGGGGTGACTGAGAAGCAGT | (SEQ ID NO: 1) | 386 |
| | AAGCTCTGTCGGCTCTGGGA | (SEQ ID NO: 2) | |
| LXRα | GTGGGGGTGACTGAGAAGCAGT | (SEQ ID NO: 1) | 410 |
| | GCCCTTTTTCCGCTTTTGTG | (SEQ ID NO: 3) | |
| LXRβ | ATCGCCGGTGCAGTCATGA | (SEQ ID NO: 4) | 353 |
| | TGGAAGCCCGAGGCCTTGTC | (SEQ ID NO: 5) | |
| LXRβ | ATCGCCGGTGCAGTCATGA | (SEQ ID NO: 4) | 355 |
| | AGTGGAAGCCCGAGGCCTTG | (SEQ ID NO: 6) | |

Neurospheres were prepared from the LVW as stated above. 3 days after the first split, the neurospheres were harvested and total RNA isolated using Qiagen's (Hilden, Germany) RNeasy Mini Kit according to the manufacturer's instructions. Life Technology's (Gaithersburg, MD) One-Step RT-PCR Kit was used to detect the presence of LXRα or LXRβ mRNA. Briefly, 100 ng of total RNA was used in each reaction, with an annealing temperature of 55° C. and 37 cycles of amplification. To ensure that genomic contamination of the total RNA did not give rise to false positive results, an identical reaction in which the RT-taq polymerase mix was replaced by taq polymerase alone and was run in parallel with the experimental RT-PCR (genomic control). The reactions were electrophoresed on a 1.5% agarose gel containing ethidium bromide and the bands visualized under UV light.

Results:

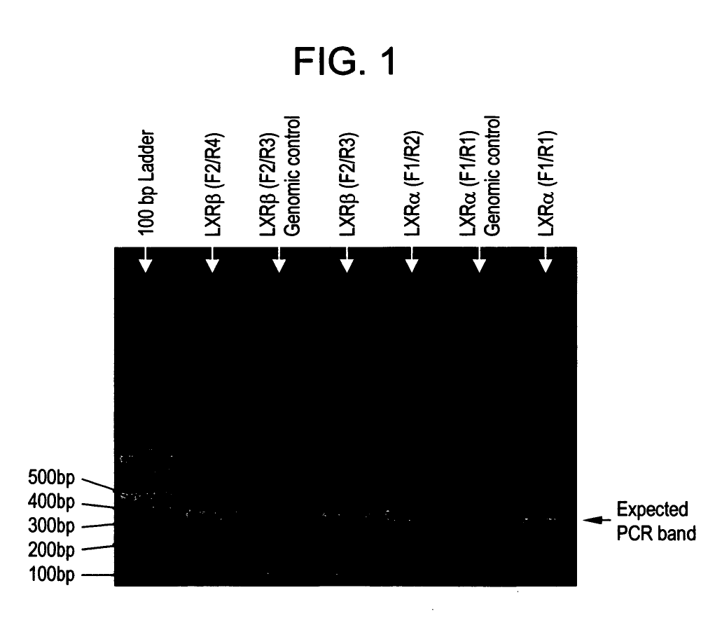

Bands corresponding to the estimated length of PCR products of the desired genes were obtained and were cloned into the cloning vector pGEM-Teasy and sequenced to verify their identity (FIG. 1). LXR transcript exist in adult NSC.

D. Western Blot

One million adult mouse neurospheres was cultured as described above, centrifugated, washed two times with PBS (Gibco) and lysed with 200 ul lysis buffer containing PBS, 0,1% Triton X-100, lmM EDTA, ltabl Protease inhibitor cocktail (Roche) per 5ml lysisbuffer. The sample were run on a 4-12% Bis-Tris gel (Novex) under reduced conditions with MOPS buffer (Novex) and blotted on to a Hybond ECL nitrocellulose membrane (Amersham Biotech). The membrane were blocked in 5% ECL-Block (Amersham Biotech) in PBS, 0,1% Tween 20 (Sigrna). The membrane were labelled with rabbit anti LXRα and β (1:5000 Chemicon). The primary antibody was detected using a secondary antibody anti-rabbit-HRP (1:10000 Amersham Biotech) and ECL Plus, Western Blot Detection Kit (Amersham Biotech). All antibodies were diluted in PBS with 0,1% Triton X-100, 1% ECL Block. The signal was captured on ECL Hyper Film (Amersham Biotech).

Results:

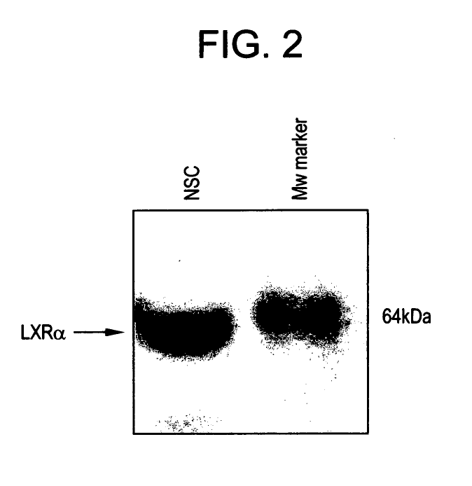

Bands corresponding to the estimated size of LXRα were identified (FIG. 2). LXR protein exist in adult NSC.

E. Detection of LXRs in proprietary mouse brain libraries:

The proprietary library sequences were analyzed by using Locus Link or the BLAST program at NCBI.

Results:

Sequences (No1, -4) corresponding to LXRβ were detected within the libraries (see Example 4).

### Example 2

### LXRα and LXRβ stimulation by LXR agonist TO-901317, 5-Cholesten-24(s), 25-epoxy-3b-ol mediates adult mouse NSC proliferation or survival in vitro

Methods

A. Mouse Neurosphere Cultures

The anterior lateral wall of the lateral ventricle of 5-6 week old mice was enzymatically dissociated in 0.8mg/ml hyaluronidase and 0.5 mg/ml trypsin in DMEM containing 4.5 mg/ml glucose and 80units/ml DNase at 37° C. for 20 min. The cells were gently triturated and mixed with three volumes of Neurosphere medium (DMEM/F12, B27 supplement, 12.5 mM HEPES pH7.4) containing 20 ng/ml EGF (unless otherwise stated), 100units/ml penicillin and 100 μg/ml streptomycin. After passing through a 70 μm strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split neurosphere cultures, neurospheres were collected by centrifugation at 160×g for 5 min. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, resuspended in fresh Neurosphere medium supplemented with 20 ng/ml EGF and 1 nM bFGF plated out and incubated at 37° C.

Chemicals for dissociation of tissue; Trypsin, Hyaluronidase and DNase were from SIGMA. Medium (DMEM 4,5 mg/ml glucose, and DMEM/F12), B27 supplement and Trypsin/EDTA were from GIBCO. All plastic ware were purchased from ComingCostar. EGF for cell cultures was from BD Biosciences.

B. Intracellular ATP Assay

Intracellular ATP levels have previously been shown to correlate to cell number (Crouch, Kozlowski et al. 1993). Mouse neurospheres, cultured as described above, from passage 2, were seeded in DMEM/F12 supplemented with B27 into a 96-well plate as single cells (10000 cells/well) to the substances to be measured were added at the concentrations indicated. After 3 days incubation, intracellular ATP was measured using the ATP-SL kit from BioThema, Sweden, according to the manufacturer's instructions. The LXR agonists: 5-Cholesten-24(s), 25-epoxy-3b-ol were purchased from Steraloids (no C6640-000): TO-901317 were purchased from Sigma-Aldrich (no T2320). The RXR agonist cis-4,7,10,13,16,19-Docosahexaenoic acid (DHA) were purchased from Sigma-Aldrich (no 43938).

Results

LXR Agonists Stimulates Adult Mouse NSC Proliferation In Vitro

To determine the effect of LXR agonists on neural stem cells in culture, mouse adult neural stem cells derived from the lateral ventricle wall of the brain, expanded in EGF as neurospheres followed by enzymatic dissociation using trypsin, were cultured in Neurosphere medium supplemented with varying concentrations of TO-901317, 5-Cholesten-24(s), 25-epoxy-3b-ol or cis-4,7,10,13,16,19-Docosahexaenoic acid (DHA) under non-adherent conditions, for 3 days. To ascertain whether there was an increase in cell number of treated cells relative to control cells, an assay measuring intracellular ATP levels, shown previously to correlate with cell number (Crouch, Kozlowski et al. 1993), was employed.

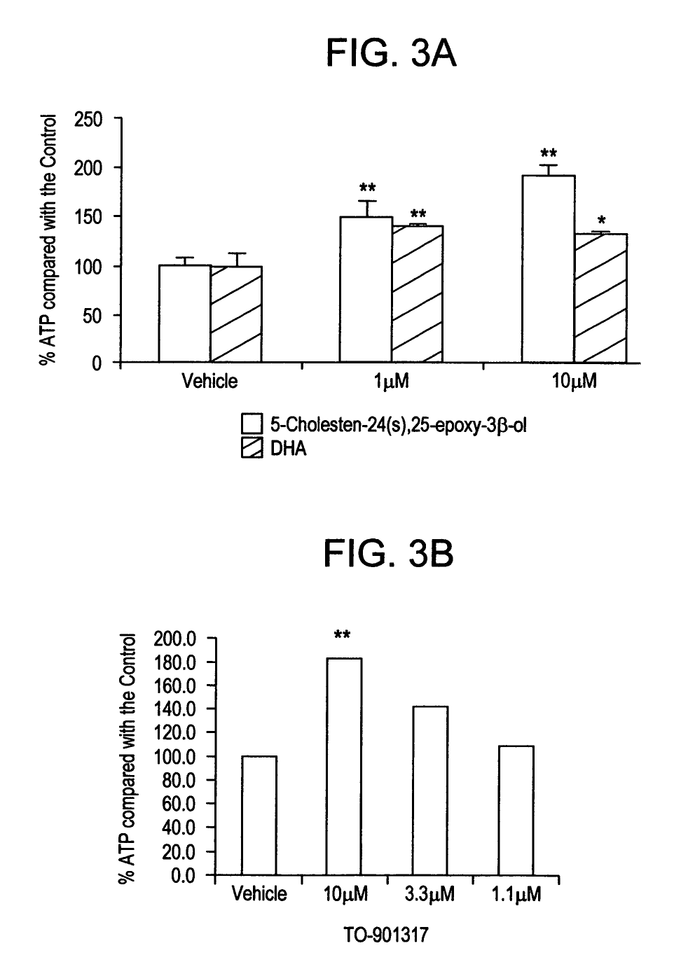

FIG. 3A and 3B shows a statistically significant increase in intracellular ATP levels, and hence cell number. Values were normalized to the vehicle (100%). The data representing the mean +/− s.e.m. of four individual wells in one experiment. The experiment was independently repeated at least two times. **, P<0.001; *P<0.05 (student's t test) as compared with the vehicle control.

### Example 3

### LXR Agonist TO-901317 and Adult Mouse NSC Proliferation In Vivo Methods

A. Intracerebroventricular Infusion of TO-901317 Infusion

10 week old male Wistar rats maintained on a 12hr light/dark cycle with food and water ad libidum, were infused in the right lateral ventricle with TO-901317 (Sigma-Aldrich), using an Alzet pump (2002), 14 days at a daily dose of 0.6 nmol/animal/day (infused at a rate of 0.5 μl/hr; 50 μM solution). Bromodeoxyuridine (BrdU) (50 mg/ml) was also included in the infusion vehicle (0.9% saline containing 1 mg/ml mouse serum albumin (Sigma)) to enable measurement of proliferation by quantitation of BrdU incorporation in the DNA. Animals were sacrificed at day 14, perfused with PBS and the brains removed and frozen at −70° C. prior to sectioning for immumohistochemical analysis.

B. Oral Administration of TO-901317/BrdU Infusion

C57BL/6J male mice were housed under controlled temperature and humidity and had free access to water and chow. 40 mice were divided into 5 groups (n=8). Group 1 received chow mixed with 0.025% T0901317 (Sigma-Aldrich) ad libitum for 10 days (dose previously show to be effective for brain activity of the compound). On day 7 and 8 the animals received a single injection of 5-bromodeoxyuridine, 100 mg/kg i.p., respectively and were sacrificed on day 10 by means of cervical dislocation.

Group 2 received identical treatment except that the chow in this case contained 0.0025% T0901317. Group 3 and 4 received identical treatments as groups 1 and 2, respectively, with the exception that these animals received 5-bromodeoxyuridine injections on day 1 and 2 after the initiation of the experiment and were sacrificed on day 4. Group 5 served as control and received chow without any T0901317 but identical to groups 1-4 received two daily injections of 5-bromodeoxyuridine 100 mg/kg i.p. and was sacrificed two days later. The mean daily intake was 4.25±0.13 (S.E.M.) g chow, and the mean weight of the animals 24.8±0.11 (S.E.M.) g during the cause of the experiment resulting in an average dose of 43 mg/kg/day for groups 1 and 3 (“High”), and an average dose of 4.3 mg/kg/day for groups 2 and 4 (“Low”). No difference between groups with regards to weight gain or food intake were observed. No correlation between weight gain, food intake or BrdU incorporation could be detected. (One-way ANOVA, Dunett's post-hoc test and bivariate analysis)

C. Immunohistochemistry

Brains were cut into 14-μm coronal sections using a cryostat-microtome. The sections were thawed onto pretreated slides and fixed in 4% (wt/vol) paraformaldehyde/PBS for 10 min. After washing in PBS, the sections were treated with 2M HCl at 37° C. for 30 min to increase accessibility of the anti-BrdU antibody to the cell nuclei. The sections were rinsed in PBS and transferred to blocking solution (PBS; 0.1% Tween; 10% goat serum) overnight at 4° C. Primary antibody (rat anti-BrdU, Harlan Sera Labs) was applied at 1:100 in blocking solution for 90 min at room temperature. After washing in PBS/0.1% Tween for 3×30 min, secondary biotinylated antibody (goat anti-rat, VectorLabs) was added at a 1:200 dilution in blocking solution for 60 min at room temperature. The sections were washed for 2 hours prior to treatment with Vectastain Kit (VectorLabs) according to the manufacturer's protocol. After 1 hour of washing, the BrdU-antibody complex was detected using 0.05% diaminobenzidine with 0.01% H₂O₂, and counterstained with Hematoxylin. The sections were dehydrated in a graded series of ethanol concentrations, followed by xylene and 99% ethanol, and mounted in Pertex. Sections were visualised using a Nikon Eclipse E600 microscope and pictures taken with a Spot Insight CCD camera.

D. Quantification and Statsistical Analysis

For all BrdU labeling experiments, three to six sections per animal were analyzed. BrdU-positive cells were counted along 2×250 micrometer strips of the lateral ventricle wall (including the ependymal cell layer and subventricular zone). The experimental group mean value was compared with the control group mean value. The numbers of BrdU-positive cells are calculated per area (mm²). The mean ± SEM were calculated. Differences between means were determined by one-way ANOVA.

Results

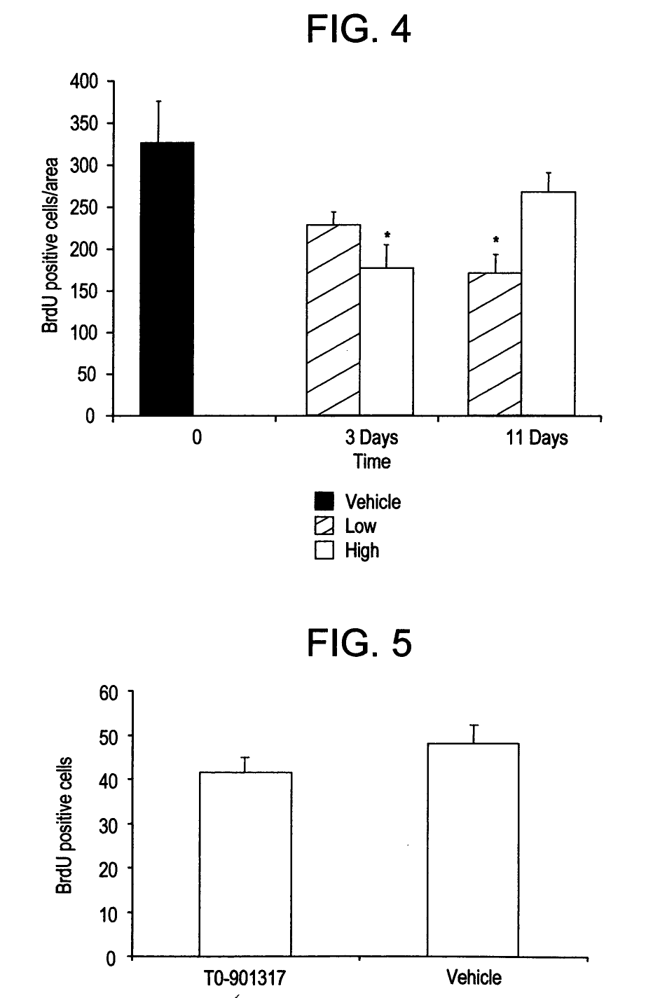

Orally administered LXR agonist TO-901317 at two different doses significantly decreased incorporation of BrdU of cells in the LVW (FIG. 4) in the mouse model.

Intracerebroventricular infusion (icv) of LXR agonist TO-901317 (calculated day concentration, assuming a 3 ml daily volume of CSF in the rat, 0.2 μM) did not significantly change incorporation of BrdU in the LVW (FIG. 5) in the rat model. A small nonsignificant decrease was observed.

Systemic TO-901317 was clearly effective at the used doses; 4.3 and 43 mg/kg/day. While local administration (icv) was ineffective at the used dose. Given the concentration-response relationship observed in vitro with cultured mouse NSC (3-5 μM in Example 2) it is likely that the effect at the dose level used in the rat icv study is indicative of the minimum dose range for this route of administration.

The in vivo results show that modulation of LXR signalling changes the rate of incorporation of BrdU indicative of a altered level of proliferation of the resident adult neural stem cells in the LVW region.

The direction of modulation of proliferation exerted by the LXR modulator used, TO-901317, is different when comparing in vitro results and in vivo results. This observation is indicative of differences in the assay paradigms and does not invalidate that the usefulness of LXR modulation of neurogenesis nor the therapeutic use of SLRMs in CNS disorders.

### Example 4

### Biopolymer Sequences

The DNA and protein sequences referenced in this patent are as listed below.

A. LXRα and LXRβ

| | Gene | NCBI sequence | |
|---|---|---|---|
| Name | Symbol | mRNA no: | NCBI protein no: |
| Homo sapiens LXRα | NR1H3 | NM_005693 | NP_005684 |
| Homo sapiens LXRβ | NR1H2 | NM_007121 | NP_009052 |
| Mus musculus LXRα | Nr1h3 | NM_013839 | NP_038867 |
| Mus musculus LXRβ | Nr1h2 | NM_009473 | NP_033499 |

>gi|113216291|ref|NM_{—}007121.1| Homo sapiens nuclear receptor subfamily 1, group H, member 2 (NR1H2), mRNA

CAAGAAGTGGCGAAGTTACCTTTGAGGGTATTTGAGTAGCGGCGGTGTGTCAGGGGCTAAAGAGGAGGACGAAGAAAAGCA
GAGCAAGGGAACCCAGGGCAACAGGAGTAGTTCACTCCGCGAGAGGCCGTCCACGAGACCCCCGCGCGCAGGCATGAGCCC
CGCCCCCCACGCATGAGCCCCGCCCCCCGCTGTTGCTTGGAGAGGGGCGGGACCTGGAGAGAGGCTGCTCCGTGACCCCACC
ATGTCCTCTCCTACCACGAGTTCCCTGGATACCCCCCTGCCTGGAAATGGCCCCCCTCAGCCTGGCGCCCCTTCTTCTTCACCC
ACTGTAAAGGAGGAGGGTCCGGAGCCGTGGCCCGGGGGTCCGGACCCTGATGTCCCAGGCACTGATGAGGCCAGCTCAGCCT
GCAGCACAGACTGGGTCATCCCAGATCCCGAAGAGGAACCAGAGCGCAAGCGAAAGAAGGGCCCAGCCCCGAAGATGCTGG
GCCACGAGCTTTGCCGTGTCTGTGGGGACAAGGCCTCCGGCTTCCACTACAACGTGCTCAGCTGCGAAGGCTGCAAGGGCTTC
TTCCGGCGCAGTGTGGTCCGTGGTGGGGCCAGGCGCTATGCCTGCCGGGGTGGCGGAACCTGCCAGATGGACGCTTTCATGC
GGCGCAAGTGCCAGCAGTGCCGGCTGCGCAAGTGCAAGGAGGCAGGGATGAGGGAGCAGTGCGTCCTTTCTGAAGAACAGA
TCCGGAAGAAGAAGATTCGGAAACAGCAGCAGCAGGAGTCACAGTCACAGTCGCAGTCACCTGTGGGGCCGCAGGGCAGCA
GCAGCTCAGCCTCTGGGCCTGGGGCTTCCCCTGGTGGATCTGAGGCAGGCAGCCAGGGCTCCGGGGAAGGCGAGGGTGTCCA
GCTAACAGCGGCTCAAGAACTAATGATCCAGCAGTTGGTGGCGGCCCAACTGCAGTGCAACAAACGCTCCTTCTCCGACCAG
CCCAAAGTCACGCCCTGGCCCCTGGGCGCAGACCCCCAGTCCCGAGATGCCCGCCAGCAACGCTTTGCCCACTTCACGGAGCT
GGCCATCATCTCAGTCCAGGAGATCGTGGACTTCGCTAAGCAAGTGCCTGGTTTCCTGCAGCTGGGCCGGGAGGACCAGATC
GCCCTCCTGAAGGCATCCACTATCGAGATCATGCTGCTAGAGACAGCCAGGCGCTACAACCACGAGACAGAGTGTATCACCT
TCTTGAAGGACTTCACCTACAGCAAGGACGACTTCCACCGTGCAGGCCTGCAGGTGGAGTTCATCAACCCCATCTTCGAGTTC
TCGCGGGCCATGCGGCGGCTGGGCCTGGACGACGCTGAGTACGCCCTGCTCATCGCCATCAACATCTTCTCGGCCGACCGGCC
CAACGTGCAGGAGCCGGGCCGCGTGGAGGCGTTGCAGCAGCCCTACGTGGAGGCGCTGCTGTCCTACACGCGCATCAAGAGG
CCGCAGGACCAGCTGCGCTTCCCGCGCATGCTCATGAAGCTGGTGAGCCTGCGCACGCTGAGCTCTGTGCACTCGGAGCAGG
TCTTCGCCTTGCGGCTCCAGGACAAGAAGCTGCCGCCTCTGCTGTCGGAGATCTGGGACGTCCACGAGTGAGGGGCTGGCCA
CCCAGCCCCACAGCCTTGCCTGACCACCCTCCAGCAGATAGACGCCGGCACCCCTTCCTCTTCCTAGGGTGGAAGGGGCCCTG
GGCGAGCCTGTAGACCTATCGGCTCTCATCCCTTGGGATAAGCCCCAGTCCAGGTCCAGGAGOCTCCCTCCCTGCCCAGCGAG
TCTTCCAGAAGGGGTGAAAGGGTTGCAGGTCCCGACCACTGACCCTTCCCGGCTGCCCTCCCTCCCCAGCTTACACCTCAAGC
CCAGCACGCAGCGTACCTTGAACAGAGGGAGGGGAGGACCCATGGCTCTCCCCCCCTAGCCCGGGAGACCAGGGGCCTTCCT
CTTCCTCTGCTTTTATTTAATAAAAATAAAAACAGAAA

>gi|50318921|ref|NM_{—}005693.1| Homo sapiens nuclear receptor subfamily 1, group H, member 3 (NR1H3), mRNA

CAGTGCCTTGGTAATGACCAGGGCTCCAGAAAGAGATGTCCTTGTGGCTGGGGGCCCCTGTGCCTGACATTCCTCCTGACTCT
GCGGTGGAGCTGTGGAAGCCAGGCGCACAGGATGCAAGCAGCCAGGCCCAGGGAGGCAGCAGCTGCATCCTCAGAGAGGAA
GCCAGGATGCCCCACTCTGCTGGGGGTACTGCAGGGGTGGGGCTGGAGGCTGCAGAGCCCACAGCCCTGCTCACCAGGGCAG
AGCCCCCTTCAGAACCCACAGAGATCCGTCCACAAAAGCGGAAAAAGGGGCCAGCCCCCAAAATGCTGGGGAACGAGCTAT
GCAGCGTGTGTGGGGACAAGGCCTCGGGCTTCCACTACAATGTTCTGAGCTGCGAGGGCTGCAAGGGATTCTTCCGCCGCAG
CGTCATCAAGGGAGCGCACTACATCTGCCACAGTGGCGGCCACTGCCCCATGGACACCTACATGCGTCGCAAGTGCCAGGAG
TGTCGGCTTCGCAAATGCCGTCAGGCTGGCATGCGGGAGGAGTGTGTCCTGTCAGAAGAACAGATCCGCCTGAAGAAACTGA
AGCGGCAAGAGGAGGAACAGGCTCATGCCACATCCTTGCCCCCCAGGCGTTCCTCACCCCCCCAAATCCTGCCCCAGCTCAG
CCCGGAACAACTGGGCATGATCGAGAAGCTCGTCGCTGCCCAGCAACAGTGTAACCGGCGCTCCTTTTCTGACCGGCTTCGAG
TCACGCCTTGGCCCATGGCACCAGATCCCCATAGCCGGGAGGCCCGTCAGCAGCGCTTTGCCCACTTCACTGAGCTGGCCATC
GTCTCTGTGCAGGAGATAGTTGACTTTGCTAAACAGCTACCCGGCTTCCTGCAGCTCAGCCGGGAGGACCAGATTGCCCTGCT
GAAGACCTCTGCGATCGAGGTGATGCTTCTGGAGACATCTCGGAGGTACAACCCTGGGAGTGAGAGTATCACCTTCCTCAAG
GATTTCAGTTATAACCGGGAAGACTTTGCCAAAGCAGGGCTGCAAGTGGAATCATCAACCCCATCTTCGAGTTCTCCAGGGC
CATGAATGAGCTGCAACTCAATGATGCCGAGTTTGCCTTGCTCATTGCTATCAGCATCTTCTCTGCAGACCGGCCCAACGTGC
AGGACCAGCTCCAGGTGGAGAGGCTGCAGCACACATATGTGGAAGCCCTGCATGCCTACGTCTCCATCCACCATCCCCATGA
CCGACTGATGTTCCCACGGATGCTAATGAAACTGGTGAGCCTCCGGACCCTGAGCAGCGTCCACTCAGAGCAAGTGTTTGCAC
TGCGTCTGCAGGACAAAAAGCTCCCACCGCTGCTCTCTGAGATCTGGGATGTGCACGAATGACTGTTCTGTCCCCATATTTTCT
GTTTTCTTGGCCGGATGGCTGAGGCCTGGTGGCTGCCTCCTAGAAGTGGAACAGACTGAGAAGGGCAAACATTCCTGGGAGC
TGGGCAAGGAGATCCTCCCGTGGCATTAAAAGAGAGTCAAAGGGT

>gi|66785061|ref|NM_{—}009473.1 | Mus musculus nuclear receptor subfamily 1, group H, member 2 (Nr1h2), mRNA

GGCGAAGTTACTTTTGCTTTTCGCTCAGCAAGCGCTGTTGCTTCGAGCTACTCCCAGGCTTCTGAAGTTACTTCCAAAGTGCTG
TGGAGGCACAATCACCGGTGCGGACACAGAGGCAACTCTCGCCTCCCACGGCCGTTTCCAGGGCAACAGAGTCGGAGACCCC
CTGCGACCCCCCTCCCGATCGCCGGTGCAGTCATGAGCCCCGCCTCCCCCTGGTGCACGGAGAGGGGCGGGGCCTGGAACAA
GCAGGCTGCTTCGTGACCCACTATGTCTTCCCCCACAAGTTCTCTGGACACTCCCGTGCCTGGGAATGGTTCTCCTCAGCCCAG
TACCTCCGCCACGTCACCCACTATTAAGGAAGAGGGGCAGGAGACTGATCCTCCTCCAGGCTCTGAAGGGTCCAGCTCTGCCT
ACATCGTGGTCATCTTAGAGCCAGAGGATGAGCCTGAGCGCAAGCGGAAGAAGGGGCCGGCCCCGAAGATGCTGGGCCATG
AGCTGTGCCGCGTGTGCGGAGACAAGGCCTCGGGCTTCCACTACAACGTGCTCAGCTGTGAAGGCTGCAAAGGCTTCTTCCG
GCGCAGTGTGGTCCACGGTGGGGCCGGGCGCTATGCCTGTCGGGGCAGCGGAACCTGCCAGATGGATGCCTTCATGCGGCGC
AAGTGCCAGCTCTGCCGGCTGCGCAAGTGCAAGGAGGCTGGCATGCGGGAGCAGTGCGTGCTCTCTGAGGAGCAGATTCGGA
AGAAAAGGATTCAGAAGCAGCAACAGCAGCAGCCACCACCCCCATCTGAGCCAGCAGCCAGCAGCTCAGGCCGGCCAGCGG
CCTCCCCTGGCACTTCGGAAGCAAGCAGCCAGGGCTCCGGGGAAGGAGAGGGCATCCAGCTGACCGCGGCTCAGGAGCTGAT
GATCCAGCAGTTAGTTGCCGCGCAGCTGCAGTGCAACAAACGATCTTTCTCCGACCAGCCCAAAGTCACGCCCTGGCCCCTGG
GTGCAGACCCTCAGTCCCGAGATGCCCGTCAGCAACGCTTTGCCCACTTCACCGAGCTAGCCATCATCTCGGTCCAGGAGATT
GTGGACTTTGCCAAGCAGGTGCCAGGGTTCTTGCAGTTGGGCCGGGAGGACCAGATCGCCCTCCTGAAGGCGTCCACCATTG
AGATCATGTTGCTAGAAACAGCCAGACGCTACAACCACGAGACAGAATGCATCACGTTCCTGAAGGACTTCACCTACAGCAA
GGACGACTTCCACCGTGCAGGCTTGCAGGTGGAATTCATCAATCCCATCTTCGAGTTCTCGCGGGCCATGCGGCGGCTGGGCC
TGGACGATGCAGAGTATGCCTTGCTTATCGCCATCAACATCTTCTCAGCCGATCGGCCTAATGTGCAGGAGCCCAGCCGTGT
GGAGGCCCTGCAGCAGCCATACGTGGAGGCGCTCCTCTCCTACACGAGGATCAAGCGCCCACAGGACCAGCTCCGCTTCCCA
CGCATGCTCATGAAGCTGGTGAGCCTGCGCACCCTCAGCTCCGTGCACTCGGAGCAGGTCTTTGCATTGCGACTCCAGGACAA
GAAGCTGCCGCCCTTGCTGTCCGAGATCTGGGATGTGCACGAGTAGGGGCAGCCACAAGTGCCCCAGCCTTGGTGGTGTCTTC
TTGAAGATGGACTCTTCACCTCTCCTCCTGGGGTGGGAGGACATTGTCACGGCCCAGTCCCTCGGGCTCAGCCTCAAACTCAG
CGGCAGTTGGCACTAGAAGGCCCCACCCCACCCATTGAGTCTTCCAAGAGTGGTGAGGGTCACAGGTCCTAGCCTCTGACCGT
TCCCAGCTGCCCTCCCACCCACGCTTACACCTCAGCCTACCACACCATGCACCTTGAGTGGAGAGAGGTTAGGGCAGGTGGCC
CCCCACAGTTGGGAGACCACAGGCCCTCTCTTCTGCCCCTTTTATTTAATAAAAAAACAAAAATAAA

>gi|7305320|ref|NM_{—}013839.1| Mus musculus nuclear receptor subfamily 1, group H, member 3 (Nr1h3), mRNA

GGGAACGCTGACTCTGGAGGCTGCTGGGATTAGGGTGGGGGTGACTGAGAAGCAGTCCTTCTGTCAGAGCAAAGAGCCTCCA
GGGTGAGGAGAGGAAGGAGAGAGATGGAACTAGACCGGTCTGCGGGGAAACGCGACAGTTTTGGTAGAGGGACAGTGTCTT
GGTAATGTCCAGGGCTCCAGGAAGAGATGTCCTTGTGGCTGGAGGCCTCAATGCCTGATGTTTCTCCTGATTCTGCAACGGAG
TTGTGGAAGACAGAACCTCAAGATGCAGGAGACCAGGGAGGCAACACTTGCATCCTCAGGGAGGAAGCCAGGATGCCCCAG
TCAACTGGGGTTGCTTTAGGGATAGGGTTGGAGTCAGCAGAGCCTACAGCCCTGCTCCCCAGGGCAGAGACCCTCCCAGAGC
CGACAGAGCTTCGTCCACAAAAGCGGAAAAAGGGCCCAGCCCCCAAAATGCTGGGGAACGAGCTGTGCAGTGTCTGTGGGG
ACAAAGCCTCTGGCTTCCATTACAACGTGCTGAGCTGCGAGGGCTGCAAGGGATTCTTCCGCCGCAGTGTCATCAAGGGAGC
ACGCTATGTCTGCCACAGCGGTGGCCACTGCCCCATGGACACCTACATGCGGCGGAAATGCCAGGAGTGTCGACTTCGCAAA
TGCCGCCAGGCAGGCATGAGGGAGGAGTGTGTGCTGTCAGAAGAACAGATCCGCTTGAAGAAACTGAAGCGGCAAGAAGAG
GAACAGGCTCAAGCCACTTCGGTGTCCCCAAGGGTGTCCTCACCTCCTCAAGTCCTGCCACAGCTCAGCCCAGAGCAGCTGGG
CATGATCGAGAAGCTGGTGGCTGCCCAGCAACAGTGTAACAGGCGCTCCTTCTCAGACCGCCTGCGCGTCACGCCTTGGCCCA
TTGCACCCGACCCTCAGAGCCGGGAAGCCCGACAACAGCGCTTTGCCCACTTTACTGAGCTGGCCATCGTGTCCGTGCAGGAG
ATTGTTGACTTTGCCAAACAGCTCCCTGGCTTCCTACAGCTCAGCAGGGAGGACCAGATCGCCTTGCTGAAGACCTCTGCAAT
CGAGGTCATGCTTCTGGAGACGTCACGGAGGTACAACCCCGGCAGTGAGAGCATCACCTTCCTCAAGGACTTCAGTTACAAC
CGGGAAGACTTTGCCAAAGCAGGGCTGCAGGTGGAGTTCATCAACCCCATCTTTGAGTTCTCCAGAGCCATGAATGAGCTGC
AACTCAATGATGCTGAGTTTGCTCTGCTCATTGCCATCAGCATCTTCTCTGCAGACCGGCCCAACGTGCAGGACCAGCTCCAA
GTAGAGAGGCTGCAACACACATATGTGGAGGCCCTGCACGCCTACGTCTCCATCAACCACCCCCACGACCGACTGATGTTCCC
ACGGATGCTAATGAAGCTGGTGAGCCTCCGTACTTTGAGCAGCGTCCATTCAGAGCAAGTGTTTGCCCTTCGCCTGCAGGACA
AAAAGCTTCCCCCTCTGCTGTCTGAGATCTGGGATGTCCACGAGTGACTGTTTCACCGTGTCCTTTGTGTTGGCCACATGGCGA
AGGCTCACTGACTGCTTCCCACGGGTGGAGCAGACTGAGAAGGGCAGACATTCCTGGGAGCTGGGTGAAGGAGAGAGCCTTG
CGTAGCATTAAGGGAGAGTCAACAGGTTGGGTGTTTTCTGGCTGCTGGGCAGTTGGGATCTACTAACGTTGTATACCATCTGA
AGACCTTGTTGACCCAACCAAATA

B. Sequences Detected within the Library

No 1
GCACAATCACCGGTGCGGACACAGAGCTCTCGCCTCCCAC
GGCCGTTTCCAGGGCAACAGAGTCGGAGACCCCCTGCGAC
CCCCCTCCCGATCGCCGGTGCAGTCATGAGCCCCGCCTCC
CCCTGGTGCACGGAGAGGGGCGGGGCCTGGAACAAGGCTG
CTTCGTGACCCACTATGTCTTCCCCCACAAGTTCTCTGGA
CACTCCCGTGCCTGGGAATGGTTCTCCTCAGCCCAGTACC
TCCGCCACGTCACCCACTATTAAGGAAGAGGGGCAGGAGA
CTGATCCTCCTCCAGGCTCTGAAGGGTCCAGCTCTGCCTA
CATCGTGGTCATCTTAGAGCCAGAGGATGAGCCTGAGCGC
AAGCGGAAGAAGGGGCCGGCCCCGAAGATGCTGGGCCATG
AGCTGTGCCGCGTGTGCGGAGACAAGGCTTCGGGCTTCCA
CTACAACGTGCTCAGCTGTGAAGGCTGCAAAGGCTTCTTC
CGGCGCAGTGTGGTCCACGGTGGGGCCGGGCGCTATGCCT
GTCGGGGCAGCGGAACCTGCCAGATGGATGCCTTCATGCG
GCGCAAGTGCCAGCTCTGCCGGCTGCGCAAGTGCAAGGAG
GCTGGCATGCGGGAGCAGTGCGTGC
No 2
TTTCGCTCAGCAAGCGCTGTTGCTTCGAGCTACTCCCAGG
CTTCTGAAGTTACTTCCAAAGTGCTGTGGAGGCACAATCA
CCGGTGCGGACACAGAGCTCTCGCCTCCCACGGCCGTTTC
CAGGGCAACAGAGTCGGAGACCCCCTGCGACCCCCCTCCC
GATCGCCGGTGCAGTCATGAGCCCCGCCTCCCCCTGGTGC
ACGGAGAGGGGCGGGGCCTGGAACAAGCAGGCTGCTTCGT
GACCCACTATGTCTTCCCCCACAAGTTCTCTGGACACTCC
CGTGCCTGGGAATGGTTCTCCTCAGCCCAGTACCTCCGCC
ACGTCACCCACTATTAAGGAAGAGGGGCAGGAGACTGATC
CT
No 3
CAAGCGCTGTTGCTTCGAGCTACTCCCAGGCTTCTGAAGT
TACTTCCAAAGTGCTGTGGAGGCACAATCACCGCTCTCGC
CTCCCACGGCCGTTTCCAGGGCAACAGAGTCGGAGACCCC
CTGCGACCCCCCTCCCGATCGCCGGTGCAGTCATGAGCCC
CGCCTCCCCCTGGTGCACGGAGAGGGGCGGGGCCTGGAAC
AAGGCTGCTTCGTGACCCACTATGTCTTCCCCCACAAGTT
CTCTGGACACTCCCGTGCCTGGGAATGGTTCTCCTCAGCC
CAGTACCTCCGCCACGTCACCCACTATTAAGGAAGAGGGG
CAGGAGACTGATCCTCCTCCAGGCTCTGAAGGGTCCAGCT
CTGCCTACATCGTGGTCATCTTAGAGCCAGAGGATGAGCC
TGAGCGCAAGCGGAAGAAGGGGCCGGCCCCGAAGATGCTG
GGCCATGAGCTGTGCCGCGTGTGCGGAGACAAGGCTTCGG
GCTTCCACTACAACGTGCTCAGCTGTGAAGGCTGCAAAGG
CTTCTTCCGGCGCAGTGTGGTCCACGGTGGGGCCGGGCGC
TATGCCTGTCGGGGCAGCGGAACCTGCCAGATGGATGCCT
TCATGCGGCGCAAGTGCCAGCTCTGCCGGCTGCGCAAGTG
CAAGGAGGCTGGCATG
No 4
CTTCTTGAAGATGGACTCTTCACCTCTCCTCCTGGGGTGG
GAGGACATTGTCACGGCCCAGTCCCTCGGGCTCAGCCTCA
AACTCAGCGGCAGTTGGCACTAGAAGGCCCCACCCCACCC
ATTGAGTCTTCCAAGAGTGGTGAGGGTCACAGGTCCTAGC
CTCTGACCGTTCCCAGCTGCCCTCCCACCCACGCTTACAC
CTCAGCCTACCACACCATGCACCTTGAGTGGAGAGAGGTT
AGGGCAGGTGGCCCCCCACAGTTGGGAGACCACAGGCCCT
CTCTTCTGCCCCTTTTATTTAATAAAAAAACAAAANTNAA
AAAAAAAAAAA

1. A method of alleviating a symptom of a disorder of the nervous system in a patient comprising administering an LXR activator or SLRM to modulate NSC activity in vivo to a patient suffering from the disease or disorder of the nervous system.

2. The method of claim 1 wherein the NSC activity is proliferation, differentiation, migration or survival.

3. The method of claim 1 wherein the LXR activator or SLRM is administered in an amount of 0.001 ng/kg/day to 100 mg/kg/day.

4. The method of claim 1 wherein the LXR activator or SLRM is administered to achieve a target tissue concentration of 0.01 nM to 50 μM.

5. The method of claim 4 wherein the target tissue is selected from the group consisting of the ventricular wall, the volume adjacent to the wall of the ventricular system, hippocampus, alveus, striatum, substantia nigra, retina, nucleus basalis of Meynert, spinal cord, thalamus, hypothalamus and cortex.

6. The method of claim 1 wherein the LXR activator or SLRM is administered by injection.

7. The method of claim 6 wherein the injection is given subcutaneously, intraperitoneally, intramusclularly, intracerebroventricularly, intraparenchymally, intrathecally intracranially, transdermally or trans-mucosally.

8. The method of claim 1 wherein the LXR activator or SLRM is administered orally.

9. The method of claim 1 wherein the disease or disorder of the nervous system is selected from the group consisting of neurodegenerative disorders, NSC disorders, neural progenitor disorders, ischemic disorders, neurological traumas, affective disorders, neuropsychiatric disorders, degenerative diseases of the retina, retinal injury/trauma, cognitive performance and learning and memory disorders.

10. A method of modulating the activity of an LXR in a NSC comprising the step of exposing the cell expressing the receptor to a modulator agent, wherein the exposure induces an NSC to proliferate, differentiate, migrate or survive.

11. The method of claim 10 wherein the modulator agent is an exogenous reagent, an antibody, an affibody or a combination thereof.

12. The method of claim 10 wherein the LXR is LXRalpha or LXRbeta.

13. The method of claim 10 wherein the LXR activator- or SLRM receptor is LXRalpha or LXRbeta or any binder of LXR activator or SLRM.

14. The method of claim 11 wherein the modulator agent is selected from the group consisting of LXR activators or SLRMs.

15. The method of claim 11 wherein the modulator agent is pegylated.

16. The method of claim 11 wherein the antibody is a monoclonal or a polyclonal antibody.

17. The method of claim 10 wherein the NSC is derived from fetal brain, adult brain, neural cell culture or a neurosphere.

18. The method of claim 10 wherein the NSC is derived from tissue enclosed by dura mater, peripheral nerves or ganglia.

19. The method of claim 10 wherein the NSC is derived from stem cells originating from a tissue selected from the group consisting of pancreas, skin, muscle, adult bone marrow, liver, umbilical cord tissue, umbilical cord blood, retina and perivascular tissues.

20. A method for stimulating mammalian adult NSC proliferation or neurogenesis comprising the step of contacting a cell population comprising mammalian adult NSC to a agent selected from the group consisting of LXR activators or SLRMs to form a treated NSC, wherein the treated NSC cell shows improved proliferation or neurogenesis compared to untreated cells.

21. The method of claim 20 wherein the NSC is derived from lateral ventricle wall of a mammalian brain.

22. The method of claim 20 wherein the NSC is derived from stem cells originating from a tissue selected from the group consisting of pancreas, skin, muscle, adult bone marrow, liver, umbilical cord tissue, umbilical cord blood, retina and perivascular tissues.

23. The method of claim 20 wherein the treated NSC shows improved differentiation, survival or migration compared to untreated cells.

24. A method for synergistically stimulating mammalian adult NSC proliferation or neurogenesis comprising the step of contacting a cell population comprising mammalian adult neural stem cells to a growth factor and an agent selected from the group consisting of LXR activators or SLRMs.

25. The method of claim 24, wherein the stimulation of mammalian adult NSC proliferation is greater than stimulation by the growth factor or stimulation by the agent alone.

26. The method of claim 24, wherein the stimulation of mammalian adult NSC proliferation is greater than the sum of stimulation by growth factor and stimulation by the agent.

27. The method of claim 24 wherein the growth factor is EGF.

28. A method for stimulating mammalian adult NSC proliferation comprising the step of contacting a cell population comprising mammalian adult NSC to VEGF or PDGF and an agent selected from the group consisting of LXR activators or SLRMs.

29. A method for inducing NSC proliferation comprising the step of modulating intracellular CREB phosphorylation.

30. The method of claim 29 wherein the step of increasing intracellualar CREB phosphorylation involves contacting the NSC with an agent selected from the group consisting of LXR activators or SLRMs.

31. A method for inducing NSC proliferation comprising the step of modulating intracellular AP-1 transcription.

32. The method of claim 31 wherein the step of modulating intracellular AP-1 transcription involves contacting the NSC with an agent selected from the group consisting of LXR activators or SLRMs.

33. A method for inducing NSC proliferation comprising the step of modulating intracellular protein kinase C activity.

34. The method of claim 33 wherein the step of increasing intracellular protein kinase C activity involves contacting the NSC with an agent selected from the group consisting of LXR activators or SLRMs.

35. A method for stimulating survival of mammalian adult NSC progeny comprising the step of modulating intracellular CREB phosphorylation in the mammalian adult NSC progeny.

36. The method of claim 35 wherein the step of modulating intracellular CREB phosphorylation comprises contacting the NSC with an agent selected from the group consisting of LXR activators or SLRMs.

37. A method for stimulating primary adult mammalian NSC to proliferate to form neurospheres comprising contacting the cell with an agent selected from the group consisting of LXR activators or SLRMs to produce a proliferating NSC.

38. A method for inducing the in situ proliferation, differentiation, migration or survival of an NSC located in the neural tissue of a mammal, the method comprising administering a therapeutically effective amount of LXR activators or SLRMs to the neural tissue to induce the proliferation, differentiation, migration or survival of the cell.

39. A method for accelerating the growth of an NSC in a desired target tissue in a subject, comprising:
(a) transfecting the target tissue with an expression vector containing an open reading frame encoding LXR genes in a therapeutically effective amount;
(b) expressing the open reading frame to produce a protein in the target tissue.

40. The method of claim 39 wherein the transfecting step involves administration of the expression vector by injection.

41. The method of claim 39 wherein the expression vector is a non-viral expression vector encapsulated in a liposome.

42. A method of enhancing neurogenesis in a patient suffering from a central nervous system disorder comprising the step of infusing LXR activators or SLRMs into the patient.

43. The method of claim 42 wherein the infusion is selected from the group consisting of intraventricular, intravenous, subcutaneous and intraarterial infusion.

44. A method of alleviating a symptom of a central nervous system disorder in a patient comprising the step of infusing LXR activators or SLRMs into the patient.

45. A method for producing a cell population enriched for human NSC, comprising:
(a) contacting a cell population containing NSC with a reagent that recognizes a determinant on an LXR;
(b) selecting for cells in which there is contact between the reagent and the determinant on the surface of the cells of step (a) to produce a population highly enriched for central nervous system stem cells.

46. The method of claim 45 wherein the reagent is selected from the group consisting of a small molecule, a peptide, an antibody and an affibody.

47. The method of claim 45 wherein the population containing NSC are obtained from neural tissue.

48. The method of claim 45 wherein the cell population is derived from whole mammalian fetal brain or whole mammalian adult brain.

49. The method of claim 45 wherein the human NSCs are derived from stem cells originating from a tissue selected from the group consisting of pancreas, skin, muscle, adult bone marrow, liver, umbilical cord tissue, umbilical cord blood, retina and perivascular tissue.

50. An in vitro cell culture comprising a cell population generated by the method of claim 45 wherein the cell population is enriched for cells expressing receptors selected from the group consisting of LXRalpha or LXRbeta.

51. A method for alleviating a symptom of a central nervous system disorder comprising administering the population of claim 50 to a mammal in need thereof.

52. A method of reducing a symptom of a central nervous system disorder in a patient comprising the step of administering into the spinal cord of the subject a composition comprising
(a) a population of isolated NSCs obtained from fetal or adult tissue; and
(b) LXR activators or SLRMs;
whereby the symptom is reduced.

53. A method for alleviating a symptom of a disease or disorder of the nervous system in a patient comprising the steps of:
(a) providing a population of NSC;
(b) suspending the NSC in a solution comprising LXR activators or SLRMs to generate a cell suspension; and
(c) delivering the cell suspension to an injection site in the nervous system of the patient to alleviate the symptom.

54. The method of claim 53 further comprising the step of administering to the injection site a growth factor for a period of time before the step of delivering the cell suspension.

55. The method of claim 53 further comprising the step of administering to the injection site a growth factor after the delivering step.

56. A method for transplanting a population of cells enriched for human NSC, comprising:
(a) contacting a population containing NSC with a reagent that recognizes a determinant on an LXR;
(b) selecting for cells in which there is contact between the reagent and the determinant on the surface of the cells of step (a), to produce a population highly enriched for central nervous system stem cells; and
(c) implanting the selected cells of step (b) into a non-human mammal.

57. A method of modulating a LXR activators or SLRMs of an NSC comprising the step of contacting the cell expressing the receptor to exogenous reagent, antibody, or affibody, wherein the exposure induces the NSC to proliferate, differentiate, migrate or survive.

58. The method of claim 57 wherein the NSC is derived from fetal brain, adult brain, neural cell culture or a neurosphere.

59. A method of determining an isolated candidate LXR modulator compound for its ability to modulate NSC activity comprising the steps of:
(a) administering the isolated candidate compound to a non-human mammal; and
(b) determining if the candidate compound has an effect on modulating the NSC activity in the non-human mammal.

60. The method of claim 59 wherein the determining step comprises comparing the neurological effects of the non-human mammal with a referenced non-human mammal not administered the candidate compound.

61. The method of claim 59 wherein the NSC activity is proliferation, differentiation, migration or survival.

62. The method of claim 59 wherein the LXR modulator is administered by injection.

63. The method of claim 62 wherein the injection is given subcutaneously, intraperitoneally, intramuscluarly, intracerebroventricularly, intraparenchymally, intrathecally intracranially, transdermally or trans-mucosally.

64. The method of claim 62 wherein the LXR modulator is administered via peptide fusion or micelle delivery.
